(19) United States
(12) Patent Application Publication
(10) Pub. No.: US 2024/0352038 A1
(43) Pub. Date: Oct. 24, 2024

(54) CRYSTALLINE FORMS OF RAS INHIBITORS, COMPOSITIONS CONTAINING THE SAME, AND METHODS OF USE THEREOF

(71) Applicant: Revolution Medicines, Inc., Redwood City, CA (US)

(72) Inventors: Shaoling LI, Sunnyvale, CA (US); Suresh Manthati, Sunnyvale, CA (US); Kang-Jye Chou, Foster City, CA (US); Jun Huang, San Diego, CA (US); Xiaojun Huang, San Jose, CA (US)

(21) Appl. No.: 18/634,289

(22) Filed: Apr. 12, 2024

Related U.S. Application Data
(60) Provisional application No. 63/459,403, filed on Apr. 14, 2023.

(51) Int. Cl. C07D 513/22; A61K 31/5025

(52) U.S. Cl. CPC C07D 513/22; A61K 31/5025

(57) ABSTRACT

The disclosure features crystalline forms of Ras inhibitors, pharmaceutical compositions thereof, and their uses in the treatment of cancers.

### BACKGROUND

The vast majority of small molecule drugs act by binding a functionally important pocket on a target protein, thereby modulating the activity of that protein. For example, cholesterol-lowering drugs known as statins bind the enzyme active site of HMG-COA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras (ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61K) of Ras are also responsible for oncogenic activity in some cancers.

Despite extensive drug discovery efforts against Ras during the last several decades, only two agents targeting Ras have been approved in the U.S.: sotorasib and adagrasib, each targeting K-Ras^{G12C}. Additional efforts are needed to uncover additional medicines for cancers driven by the various Ras mutations.

### SUMMARY

The invention features crystalline forms of compound useful for the treatment of a disease or condition (e.g., cancer, Ras protein-related disorder).

In an aspect, this disclosure describes a crystalline form of Compound A:

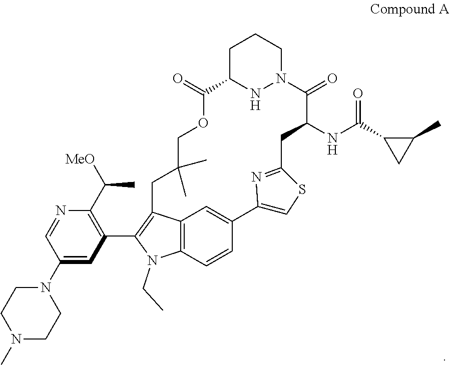

In some embodiments, the crystalline form of Compound A or the solvate thereof is selected from Form 1, Form 2, Form 3, Form 4, Form 5, Form 6, Form 7, or Form 8. In some embodiments, the crystalline form of Compound A or the solvate thereof is Form 1. In some embodiments, the crystalline form of Compound A or the solvate thereof is Form 5.

In an aspect, the invention features a crystalline hydrate of Compound A:

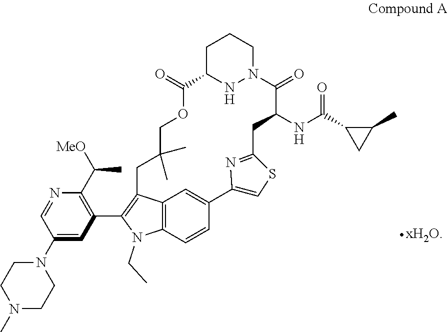

In some embodiments, the crystalline Form 1 of Compound A, or a solvate thereof, has at least one peak at diffraction angle 2θ (°) of 5.2±0.5, 8.4±0.5, or 10.0±0.5 as measured by X-ray diffractometry by irradiation with Cu Kα X-rays or calculated from X-ray diffractometry. In some embodiments, the crystalline Form 1 of Compound A, or a solvate thereof, has peaks at diffraction angle 2θ (°) of 5.2±0.5, 8.4±0.5, and 10.0±0.5 as measured by X-ray diffractometry by irradiation with Cu Kα X-rays or calculated from X-ray diffractometry. In some embodiments, the crystalline Form 1 of Compound A, or a solvate thereof, having peaks at diffraction angle 2θ (°) of 10.7±0.5, 14.4±0.5, and 16.7±0.5 as measured by X-ray diffractometry by irradiation with Cu Kα X-rays or calculated from X-ray diffractometry. In some embodiments, the crystalline Form 1 of Compound A, or a solvate thereof, has peaks at diffraction angle 2θ (°) of 5.2±0.5, 8.4±0.5, 10.0±0.5, 10.7±0.5, 14.4±0.5, and 16.7±0.5 as measured by X-ray diffractometry by irradiation with Cu Kα X-rays or calculated from X-ray diffractometry. In some embodiments, the crystalline Form 1 of Compound A, or a solvate thereof, has peaks at diffraction angle 2θ (°) of 17.0±0.5, 18.4±0.5, and 20.5±0.5 as measured by X-ray diffractometry by irradiation with Cu Kα X-rays or calculated from X-ray diffractometry. In some embodiments, the crystalline Form 1 of Compound A, or a solvate thereof, has peaks at diffraction angle 2θ (°) of 5.2±0.5, 8.4±0.5, 10.0±0.5, 10.7±0.5, 14.4±0.5, 16.7±0.5, 17.0±0.5, 18.4±0.5, and 20.5±0.5 as measured by X-ray diffractometry by irradiation with Cu Kα X-rays or calculated from X-ray diffractometry. In some embodiments, the crystalline Form 1 of Compound A, or a solvate thereof, has the X-ray powder diffractogram as shown in FIG. 1.

In some embodiments, the crystalline Form 1 of Compound A is a hydrate solvate. In some embodiments, the crystalline Form 1 of Compound A is a tri-hydrate. In some embodiments, the crystalline Form 1 of Compound A is a mixed methanol and water solvate.

In some embodiments, the crystalline Form 1 of Compound A is a mixed methanol and water solvate and is further characterized by a unit cell with parameters a=10.8182 Å, b=13.3787 Å, c=17.6881 Å, α=88.4427°, β=79.5367°, γ=82.0377° and V=2493.21 Å³.

In some embodiments, the crystalline Form 1 of Compound A, or a solvate thereof, has an endothermic onset at 66.8° C.±0.5 and 95.8° C.±0.5 in differential scanning calorimetry (DSC) profile. In some embodiments, the crystalline Form 1 of Compound A, or a solvate thereof, has a DSC thermogram shown in FIG. 3. In some embodiments, the crystalline Form 1 of Compound A, or a solvate thereof, exhibits a weight loss of 6.0%±0.5 (w/w) between ambient and 100.0° C.±0.5 in a thermogravimetric analysis (TGA) profile. In some embodiments, the crystalline Form 1 of Compound A, or a solvate thereof, has a TGA graph shown in FIG. 4.

In an aspect, the invention features a crystalline Form 5 of Compound A, or a solvate thereof, having at least one peak at diffraction angle 2θ (°) of 14.3±0.5, 16.8±0.5, or 20.5±0.5 as measured by X-ray diffractometry by irradiation with Cu Kα X-rays or calculated from X-ray diffractometry. In some embodiments, the crystalline Form 5 of Compound A, or a solvate thereof, has peaks at diffraction angle 2θ (°) of 14.3±0.5, 16.8±0.5, and 20.5±0.5 as measured by X-ray diffractometry by irradiation with Cu Kα X-rays or calculated from X-ray diffractometry. In some embodiments, the crystalline Form 5 of Compound A, or a solvate thereof, has peaks at diffraction angle 2θ (°) of 5.2±0.5, 17.0±0.5, and 20.3±0.5 as measured by X-ray diffractometry by irradiation with Cu Kα X-rays or calculated from X-ray diffractometry. In some embodiments, the crystalline Form 5 of Compound A, or a solvate thereof, has peaks at diffraction angle 2θ (°) of 5.2±0.5, 14.3±0.5, 16.8±0.5, 17.0±0.5, 20.3±0.5, and 20.5±0.5 as measured by X-ray diffractometry by irradiation with Cu Kα X-rays or calculated from X-ray diffractometry. In some embodiments, the crystalline Form 5 of Compound A, or a solvate thereof, has peaks at diffraction angle 2θ (°) of 16.3±0.5, 17.1±0.5, and 18.2±0.5 as measured by X-ray diffractometry by irradiation with Cu Kα X-rays or calculated from X-ray diffractometry. In some embodiments, the crystalline Form 5 of Compound A, or a solvate thereof, has peaks at diffraction angle 2θ (°) of 5.2±0.5, 14.3±0.5, 16.3±0.5, 16.8±0.5, 17.0±0.5, 17.1±0.5, 18.2±0.5, 20.3±0.5, and 20.5±0.5 as measured by X-ray diffractometry by irradiation with Cu Kα X-rays or calculated from X-ray diffractometry. In some embodiments, the crystalline Form 5 of Compound A, or a solvate thereof, has the X-ray powder diffractogram as shown in FIG. 18. In some embodiments, the solvate is a hydrate.

In some embodiments, the crystalline Form 5 of Compound A, or a solvate thereof, has an endothermic onset at 32.1° C.±0.5 and 81.2° C.±0.5 in differential scanning calorimetry (DSC) profile. In some embodiments, the crystalline Form 5 of Compound A, or a solvate thereof, has a DSC thermogram shown in FIG. 19. In some embodiments, the crystalline Form 5 of Compound A, or a solvate thereof, exhibits a weight loss of 4.8%±0.5 (w/w) between ambient and 150.0° C.±0.5 in a thermogravimetric analysis (TGA) profile. In some embodiments, the crystalline Form 5 of Compound A, or a solvate thereof, has a TGA graph shown in FIG. 20.

In an aspect, the invention features a crystalline form of a tosylate salt of Compound A, or a solvate thereof:

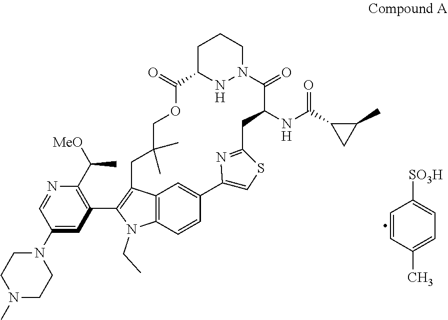

In some embodiments, the crystalline form has at least one peak at diffraction angle 2θ (°) of 5.9±0.5, 9.4±0.5, or 9.8±0.5 as measured by X-ray diffractometry by irradiation with Cu Kα X-rays or calculated from X-ray diffractometry. In some embodiments, the crystalline salt form of Compound A, or a solvate thereof, has peaks at diffraction angle 2θ (°) of 5.9±0.5, 9.4±0.5, and 9.8±0.5 as measured by X-ray diffractometry by irradiation with Cu Kα X-rays or calculated from X-ray diffractometry. In some embodiments, the crystalline salt form of Compound A, or a solvate thereof, has peaks at diffraction angle 2θ (°) of 10.2±0.5, 10.6±0.5, 11.0±0.5, and 16.1±0.5 as measured by X-ray diffractometry by irradiation with Cu Kα X rays or calculated from X-ray diffractometry. In some embodiments, the crystalline salt form of Compound A, or a solvate thereof, has peaks at diffraction angle 2θ (°) of 5.9±0.5, 9.4±0.5, 9.8±0.5, 10.2±0.5, 10.6±0.5, 11.0±0.5, and 16.1±0.5 as measured by X-ray diffractometry by irradiation with Cu Kα X-rays or calculated from X-ray diffractometry. In some embodiments, the crystalline salt form of Compound A, or a solvate thereof, has peaks at diffraction angle 2θ (°) of 12.6±0.5, 12.8±0.5, 14.2±0.5, and 17.2±0.5 as measured by X-ray diffractometry by irradiation with Cu Kα X-rays or calculated from X-ray diffractometry. In some embodiments, the crystalline salt form of Compound A, or a solvate thereof, has peaks at diffraction angle 2θ (°) of 5.9±0.5, 9.4±0.5, 9.8±0.5, 10.2±0.5, 10.6±0.5, 11.0±0.5, 12.6±0.5, 12.8±0.5, 14.2±0.5, 16.1±0.5, and 17.2±0.5 as measured by X-ray diffractometry by irradiation with Cu Kα X-rays or calculated from X-ray diffractometry. In some embodiments, the crystalline salt form of Compound A, or a solvate thereof, has the X-ray powder diffractogram as shown in FIG. 6. In some embodiments, the crystalline salt form of Compound A is a hydrate. In some embodiments, the crystalline salt form of Compound A, is a hemi-hydrate. In some embodiments, the crystalline salt form of Compound A, or a solvate thereof, has an endothermic onset at 99.5° C.±0.5, 230.7° C.±0.5, and 262.4° C.±0.5 in differential scanning calorimetry (DSC) profile.

In some embodiments, the crystalline salt form of Compound A, or a solvate thereof, has a DSC thermogram shown in FIG. 7. In some embodiments, the crystalline salt form of Compound A, or a solvate thereof, exhibits a weight loss of 2.7%±0.5 (w/w) between ambient and 50.0° C.±0.5, or weight loss of 3.2%±0.5 (w/w) between ambient and 200.0° C.±0.5, in a thermogravimetric analysis (TGA) profile. In some embodiments, the crystalline salt form of Compound A, or a solvate thereof, has a TGA graph shown in FIG. 8.

In some embodiments, a pharmaceutical composition includes the crystalline forms of Compound A, or a solvate thereof, and a pharmaceutically acceptable carrier or excipient.

In an aspect, the invention features a method of making a crystalline Form 1 of Compound A,

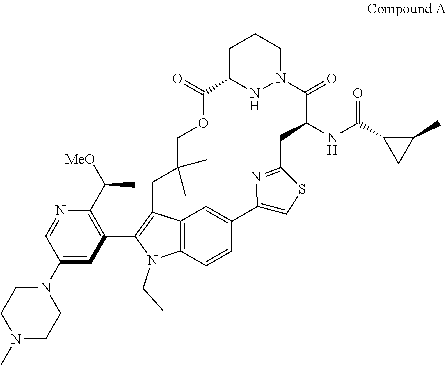

or a solvate thereof, including preparing a slurry of Compound A in a suitable solvent or buffer to precipitate crystalline solids of Form 1, isolating the crystalline solids of Form 1 from the slurry, and drying the crystalline solids of Form 1. In some embodiments, the suitable solvent is a mixture of methanol and water. In some embodiments, the suitable buffer is acetate buffer (pH 4).

In an aspect, the invention features a method of making a crystalline Form 5 of Compound A,

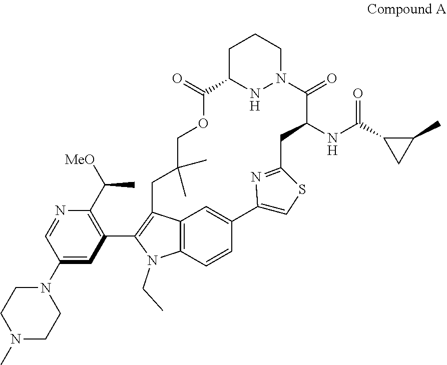

or a solvate thereof, including preparing a slurry of crystalline Form 1 of Compound A in a suitable solvent to precipitate crystalline solids of Form 5, isolating the crystalline solids of Form 5 from the slurry, and drying the crystalline solids of Form 5. In some embodiments, the suitable solvent is selected from a group consisting of a mixture of isopropyl alcohol/water and a mixture of acetone/water.

In an aspect, the invention features a method of making a crystalline Form 5 of Compound A,

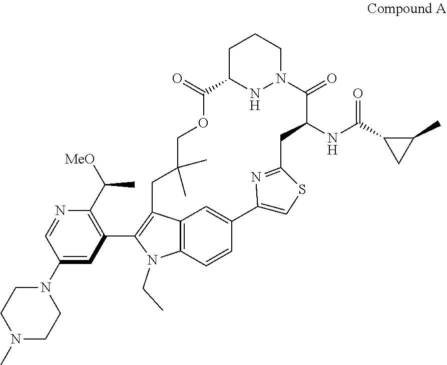

or a solvate thereof, including preparing a mixture of crystalline Form 1 of Compound A in isopropyl alcohol, precipitating crystalline solids of Form 5 by the addition of water, isolating the crystalline solids of Form 5 from the slurry, and drying the crystalline solids of Form 5.

In an aspect, the invention features a method of making a crystalline form of a tosylate salt of Compound A,

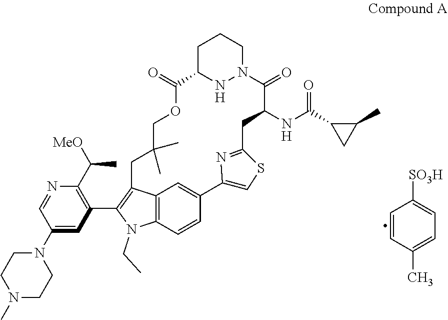

or a solvate thereof, including preparing a mixture of Compound A and 4-toluenesulfonic acid in a suitable solvent or buffer to precipitate crystalline solids of the tosylate salt of Compound A, isolating the crystalline solids of the tosylate salt of Compound A from the mixture, and drying the crystalline solids of the tosylate salt of Compound A. In some embodiments, the method includes heating the mixture prior to isolating the solids from the slurry. In some embodiments, the suitable solvent is acetonitrile, water, or a mixture of acetonitrile and water.

In some embodiments, the invention features a method of treating cancer in a subject in need thereof, and the method including administering to the subject a therapeutically effective amount of a crystalline form of Compound A, or a solvate thereof, or a pharmaceutical composition. In some embodiments, the cancer includes a Ras mutation. In some embodiments, the Ras mutation is at position 12, 13 or 61, or a combination thereof. In some embodiments, the Ras mutation is at a position selected from the group consisting of G12C, G12D, G12V, G12R, G13C, G13D, and Q61K, or a combination thereof. In some embodiments, the Ras mutation is at a position selected from the group consisting of G12D, G12V and G12R, or a combination thereof. In some embodiments, the Ras mutation is at a position selected from the group consisting of G12D and G12V, or a combination thereof. In some embodiments, the cancer is pancreatic cancer. In some embodiments, the cancer is lung cancer. In some embodiments, the cancer is non-small cell lung cancer. In some embodiments, the cancer is colorectal cancer. In some embodiments, the invention features a method of treating a Ras protein-related disorder in a subject in need thereof, and the method including administering to the subject a therapeutically effective amount of a crystalline form of Compound A, or a solvate thereof, or a pharmaceutical composition.

In some embodiments, the invention features a method of inhibiting a Ras protein in a cell, and the method including contacting the cell with an effective amount of a crystalline form of Compound A, or a solvate thereof, or a pharmaceutical composition. In some embodiments, more than one Ras protein is inhibited in the cell. In some embodiments, the cell is a cancer cell. In some embodiments, the cancer cell is a pancreatic cancer cell. In some embodiments, the cancer cell is a lung cancer cell. In some embodiments, the cancer cell is a non-small cell lung cancer cell. In some embodiments, the cancer cell is a colorectal cancer cell. In some embodiments, the Ras protein includes KRAS.

In some embodiments, the method further includes administering an additional anticancer therapy. In some embodiments, the additional anticancer therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof. In some embodiments, the second Ras inhibitor is a KRAS^{G12C} (OFF) inhibitor.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “crystalline form of the compound” and similar terms as used herein, whether explicitly noted or not, refers to Ras inhibitors described herein, including crystalline forms of a compound of Formula I, solvates, hydrates, and tautomers thereof.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

The details of one or more embodiments of the invention are set forth in the description below. Other features, objects, and advantages of the invention will be apparent from the description and from the claims.

### BRIEF DESCRIPTION OF THE DRAWINGS

FIG. 1A is an exemplary X-ray powder diffractogram of crystalline Form 1 of Compound A free base as the mixed methanol and water solvate. FIG. 1B is an exemplary X-ray powder diffractogram of crystalline Form 1 of Compound A free base as the tri-hydrate solvate.

FIG. 2 is an exemplary X-ray crystal structure of crystalline Form 1 of Compound A free base (asymmetric unit shown). Hydrogen bonds are black, dotted lines. Hydrogen atoms have been omitted for clarity.

FIG. 3 is an exemplary differential scanning calorimetry (DSC) thermogram of crystalline Form 1 of Compound A free base.

FIG. 4 is an exemplary thermogravimetric analysis (TGA) of crystalline Form 1 of Compound A free base.

FIG. 5 is an exemplary 1H NMR spectrum of crystalline Form 1 of Compound A free base.

FIG. 6 is an exemplary X-ray powder diffractogram of crystalline Compound A tosylate salt.

FIG. 7 is an exemplary DSC thermogram of crystalline Compound A tosylate salt.

FIG. 8 is an exemplary TGA of crystalline Compound A tosylate salt.

FIG. 9 is an exemplary 1H NMR spectrum of a crystalline Compound A tosylate salt.

FIG. 10 is an exemplary dynamic vapor sorption (DVS) of crystalline Compound A tosylate salt.

FIG. 11 is an exemplary X-ray powder diffractogram of crystalline Form 2 of Compound A.

FIG. 12 is an exemplary DSC thermogram of crystalline Form 2 of Compound A.

FIG. 13 is an exemplary TGA of crystalline Form 2 of Compound A.

FIG. 14 is an exemplary X-ray powder diffractogram of crystalline Form 3 of Compound A.

FIG. 15 is an exemplary X-ray powder diffractogram of crystalline Form 4 of Compound A.

FIG. 16 is an exemplary DSC thermogram of crystalline Form 4 of Compound A.

FIG. 17 is an exemplary TGA of crystalline Form 4 of Compound A.

FIG. 18 is an exemplary X-ray powder diffractogram of crystalline Form 5 of Compound A.

FIG. 19 is an exemplary DSC thermogram of crystalline Form 5 of Compound A.

FIG. 20 is an exemplary TGA of crystalline Form 5 of Compound A.

FIG. 21 is an exemplary X-ray powder diffractogram of crystalline Form 6 of Compound A.

FIG. 22 is an exemplary DSC thermogram of crystalline Form 6 of Compound A.

FIG. 23 is an exemplary TGA of crystalline Form 6 of Compound A.

FIG. 24 is an exemplary X-ray powder diffractogram of crystalline Form 7 of Compound A.

FIG. 25 is an exemplary X-ray powder diffractogram of crystalline Form 8 of Compound A.

FIG. 26 is an overlay of X-ray powder diffractograms of crystalline forms 1-8 of Compound A.

### DETAILED DESCRIPTION

### Compounds

In general, the invention provides crystalline forms of Formula I. The compound of Formula I, hereafter referred to as Compound A, is of the following structure:

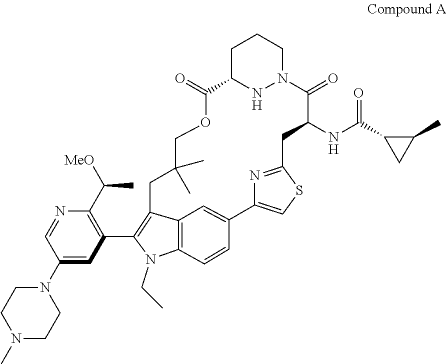

A crystalline form of Compound A may be, e.g., a free base or tosylate salt.

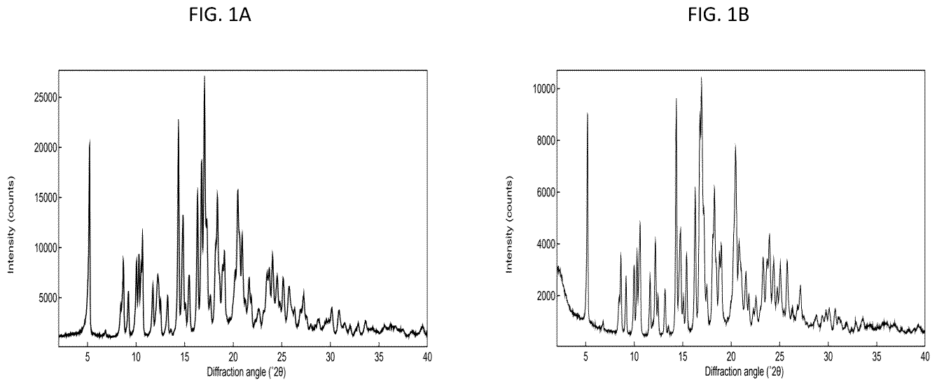

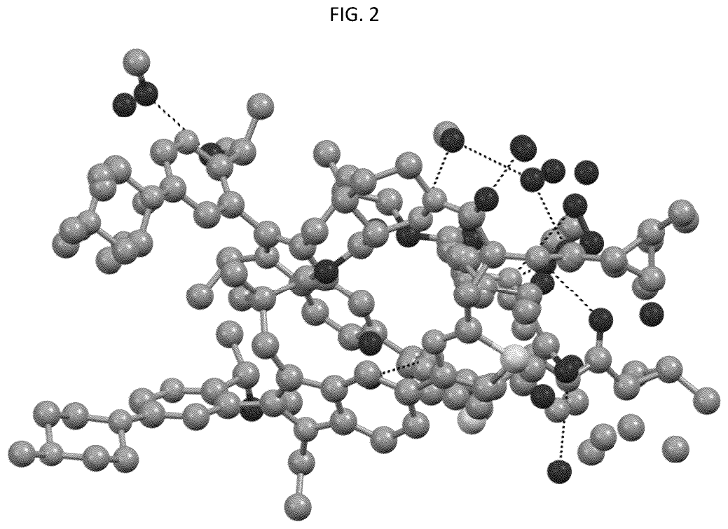

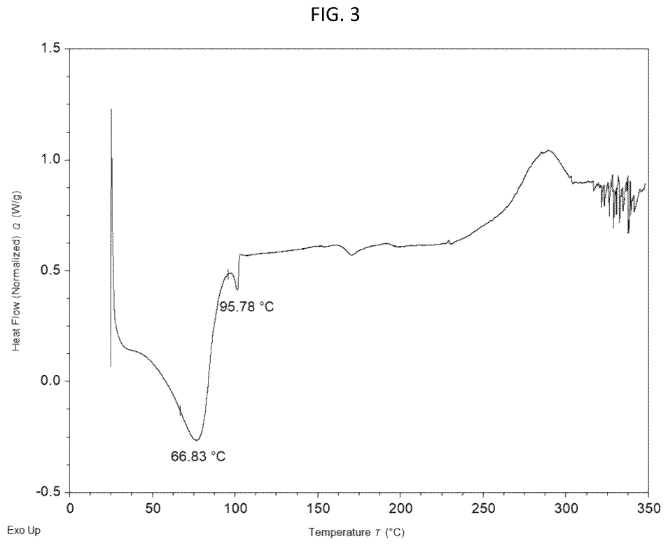

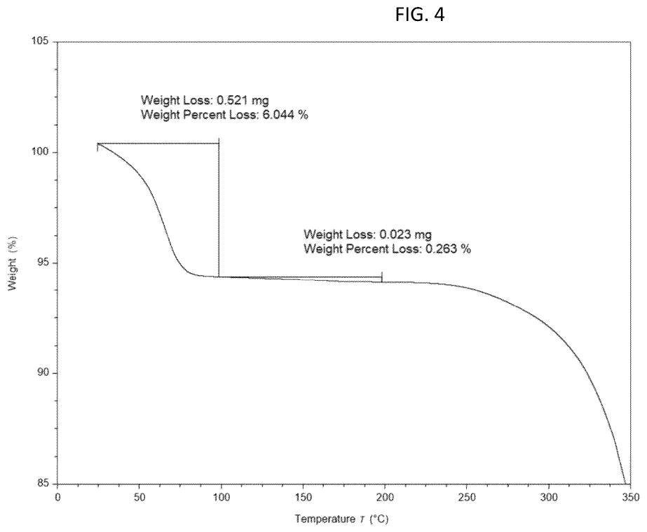

As described in the examples, a crystalline Form 1 of Compound A, or solvate thereof, may have one or more peaks at diffraction angle 2θ (°) of 5.2±0.5, 8.4±0.5, 10.0±0.5, 10.7±0.5, 14.4±0.5, 16.7±0.5, 17.0±0.5, 18.4±0.5, and 20.5±0.5 as measured by X-ray diffractometry by irradiation with Cu Kα X-rays or calculated from X-ray diffractometry. Form 1, or solvate thereof, may have an X-ray powder diffractogram shown in FIGS. 1A and 1B. Form 1 may have a crystal structure shown in FIG. 2. The Form 1 crystal structure is Compound A free base as a mixed methanol and water solvate. Form 1, or solvate thereof, may have an endothermic onset at 66.8° C.±0.5 and 95.8° C.±0.5 by differential scanning calorimetry (see FIG. 3). Form 1, or solvate thereof, may have a weight loss of 6.0%±0.5 (w/w) between ambient and 100.0° C.±0.5 in a thermogravimetric analysis profile (see FIG. 4).

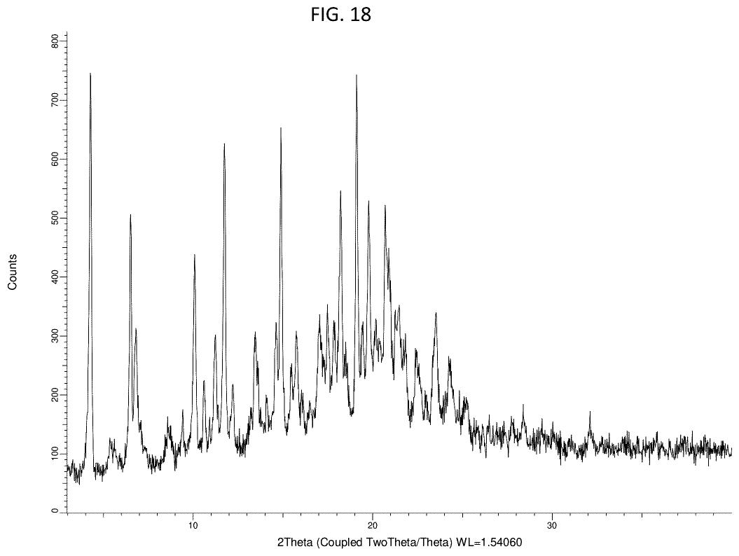

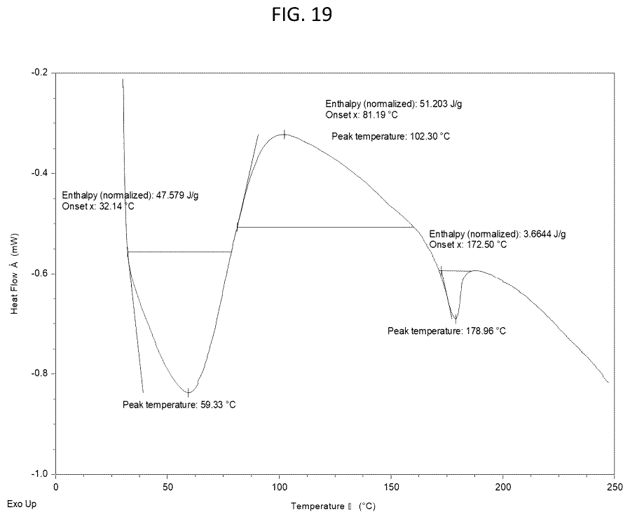

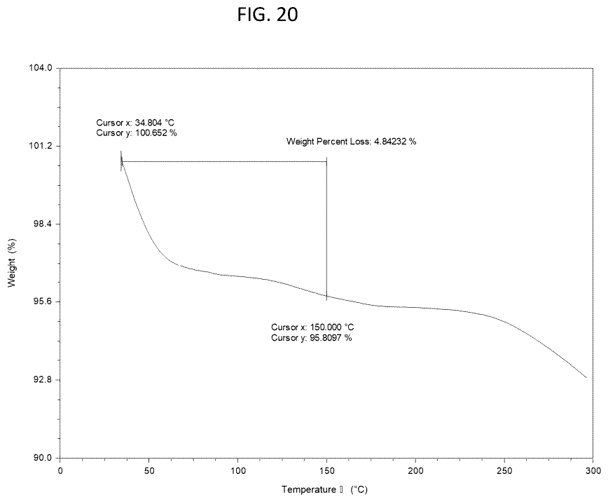

A crystalline Form 5 of Compound A, or solvate thereof, may have one or more peaks at diffraction angle 2θ (°) of 5.2±0.5, 14.3±0.5, 16.3±0.5, 16.8±0.5, 17.0±0.5, 17.1±0.5, 18.2±0.5, 20.3±0.5, and 20.5±0.5 as measured by X-ray diffractometry by irradiation with Cu Kα X-rays or calculated from X-ray diffractometry. Form 5, or solvate thereof, may have an X-ray powder diffractogram shown in FIG. 18. Form 5, or solvate thereof, may have an endothermic onset at 32.1° C.±0.5 and 81.2° C.±0.5 in differential scanning calorimetry (DSC) (see FIG. 19). Form 5, or solvate thereof, may have a weight loss of 4.8%±0.5 (w/w) between ambient and 150.0° C.±0.5 in a thermogravimetric analysis profile (see FIG. 20).

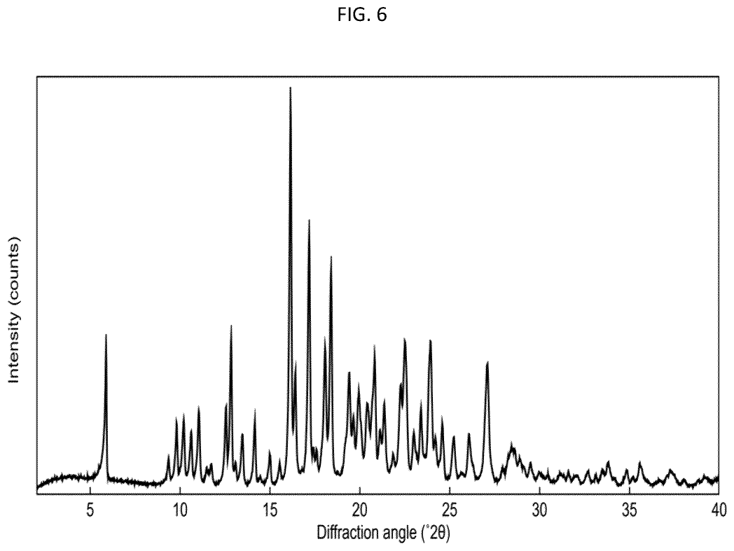

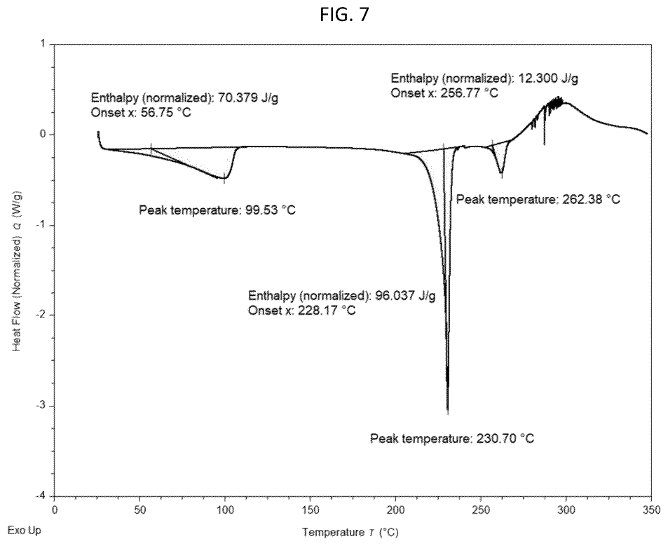

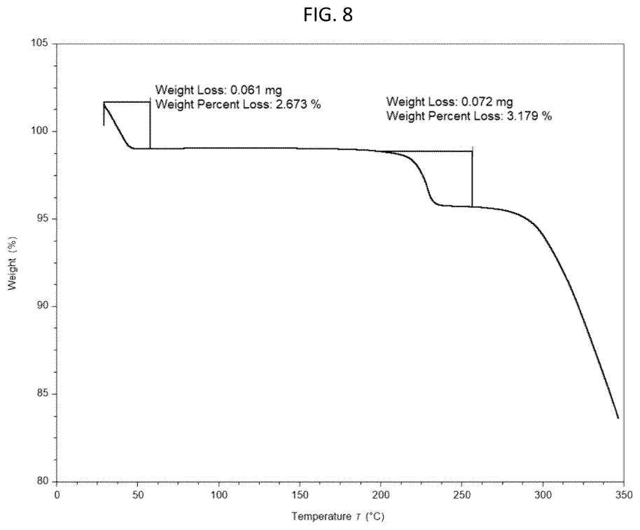

A crystalline form of Compound A tosylate salt, or solvate thereof, may have one or more peaks at diffraction angle 2θ (°) of 5.9±0.5, 9.4±0.5, 9.8±0.5, 10.2±0.5, 10.6±0.5, 11.0±0.5, 12.6±0.5, 12.8±0.5, 14.2±0.5, 16.1±0.5, and 17.2±0.5. The form, or solvate thereof, may have an X-ray powder diffractogram shown in FIG. 6. The form, or solvate thereof, may have an endothermic onset at 99.5° C.±0.5, 230.7° C.±0.5, and 262.4° C.±0.5 by DSC (see FIG. 7). The form, or solvate thereof, may have a weight loss of 2.7%±0.5 (w/w) between ambient and 50.0° C.±0.5, and a weight loss of 3.2%±0.5 (w/w) between ambient and 200.0° C.±0.5 in a thermogravimetric analysis profile (see FIG. 8).

Further provided is a method of treating cancer in a subject in need thereof, the method comprising administering to the subject a therapeutically effective amount of a crystalline compound of the present invention. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer comprises a Ras mutation, such as G12C, G12D, G12V, G12R, G13C, G13D, and Q61K. Other Ras mutations are described herein.

Further provided is a method of treating a Ras protein-related disorder in a subject in need thereof, the method comprising administering to the subject a therapeutically effective amount of a crystalline compound of the present invention.

Further provided is a method of inhibiting a Ras protein in a cell, the method comprising contacting the cell with an effective amount of a crystalline compound of the present invention. For example, the Ras protein is Ras G12C, Ras G12D, Ras G12V, Ras G12R, Ras G13C, Ras G13D, or Ras Q61K. Other Ras proteins are described herein. The cell may be a cancer cell, such as a pancreatic cancer cell, a colorectal cancer cell, a non-small cell lung cancer cell, an acute myeloid leukemia cell, a multiple myeloma cell, a thyroid gland adenocarcinoma cell, a myelodysplastic syndrome cell, or a squamous cell lung carcinoma cell. Other cancer types are described herein. The cell may be in vivo or in vitro.

In some embodiments, a method or use described herein further comprises administering an additional anti-cancer therapy. In some embodiments, the additional anti-cancer therapy is a HER2 inhibitor, an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, or a combination thereof. In some embodiments, the additional anticancer therapy is a SHP2 inhibitor. Other additional anti-cancer therapies are described herein.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. Exemplary syntheses of the compounds of the present invention are disclosed in WO2022/060836, which is incorporated herein by reference.

### Pharmaceutical Compositions and Methods of Use

The crystalline forms of compounds with which the invention is concerned are Ras inhibitors and are useful in the treatment of cancer. Accordingly, one embodiment of the present invention provides pharmaceutical compositions containing a crystalline form of the invention, and a pharmaceutically acceptable excipient, as well as methods of using the compounds of the invention to prepare such compositions.

As used herein, the term “pharmaceutical composition” refers to a compound or crystalline form, such as a crystalline compound of the present invention, formulated together with a pharmaceutically acceptable excipient.

In some embodiments, the crystalline form(s) of the compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a crystalline compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or Compound Administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a crystalline compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a crystalline compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder, or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the crystalline forms of the compounds of the invention can be formulated as pharmaceutical or veterinary compositions. Depending on the subject to be treated, the mode of administration, and the type of treatment desired, e.g., prevention, prophylaxis, or therapy, the compounds, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from 0.1% to 99%, from 5% to 90%, or from 1% to 20% of a crystalline compound of the present invention, by weight or volume. In some embodiments, crystalline forms of the compounds, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a crystalline form of Compound A, or a preparation that includes a crystalline form of Compound A described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. A formulation will generally include a diluent as well as, in some cases, adjuvants, buffers, preservatives and the like. Crystalline forms of compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, and tablets, as is understood in the art.

Each crystalline form of a compound as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first crystalline compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of crystalline compounds, or by incorporating the crystalline compound, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the crystalline forms of compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the crystalline compounds of the invention will depend on the nature of the crystalline compound, and can readily be determined by one skilled in the art. A dosage may be, for example, 0.001 mg to 2000 mg per day, 1 mg to 1000 mg per day, 5 mg to 500 mg per day, 100 mg to 1500 mg per day, 500 mg to 1500 mg per day, 500 mg to 2000 mg per day, or any range derivable therein. In some embodiments, a dosage may be 10 mg per day. In some embodiments, the dosage may be 20 mg per day. In some embodiments, the dosage may be 40 mg per day. In some embodiments, the dosage may be 80 mg per day. In some embodiments, the dosage may be 120 mg per day.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

In some embodiments, the invention discloses a method of treating a disease or disorder that is characterized by aberrant Ras activity due to a Ras mutant. In some embodiments, the disease or disorder is a cancer.

Accordingly, also provided is a method of treating cancer in a subject in need thereof, the method comprising administering to the subject a therapeutically effective amount of a crystalline form of a compound of the present invention or a pharmaceutical composition comprising such a crystalline form of a compound or salt. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a Ras protein-related disorder in a subject in need thereof, the method comprising administering to the subject a therapeutically effective amount of a crystalline compound of the present invention or a pharmaceutical composition comprising such a crystalline compound or salt.

In some embodiments, the crystalline forms of the compound of the present invention pharmaceutical compositions comprising such crystalline forms, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate, and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma, and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and Adrenal glands, for example: neuroblastoma.

In some embodiments, the Ras protein is wild type (Ras^{WT}). Accordingly, in some embodiments, a crystalline compound of the present invention is employed in a method of treating a patient having a cancer comprising a Ras^{WT} (e.g., K-Ras^{WT}, H-Ras^{WT} or N-Ras^{WT}). In some embodiments, the Ras protein is Ras amplification (e.g., K-Ras^{amp}). Accordingly, in some embodiments, a crystalline compound of the present invention is employed in a method of treating a patient having a cancer comprising a Ras^{amp} (K-Ras^{amp}, H-Ras^{amp} or N-Ras^{amp}). In some embodiments, the cancer comprises a Ras mutation, such as a Ras mutation described herein. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof;
   or a combination of any of the foregoing. In some embodiments, the cancer comprises a K-Ras mutation selected from the group consisting of G12C, G12D, G13C, G12V, G13D, G12R, G12S, Q61H, Q61K and Q61L. In some embodiments, the cancer comprises an N-Ras mutation selected from the group consisting of G12C, Q61H, Q61K, Q61L, Q61P and Q61R. In some embodiments, the cancer comprises an H-Ras mutation selected from the group consisting of Q61H and Q61L. In some embodiments, the cancer comprises a Ras mutation selected from the group consisting of G12C, G13C, G12A, G12D, G13D, G12S, G13S, G12V and G13V. In some embodiments, the cancer comprises at least two Ras mutations selected from the group consisting of G12C, G13C, G12A, G12D, G13D, G12S, G13S, G12V and G13V. In some embodiments, a crystalline compound of the present invention inhibits more than one Ras mutant. For example, a compound may inhibit both K-Ras G12C and K-Ras G13C. A compound may inhibit both N-Ras G12C and K-Ras G12C. In some embodiments, a crystalline compound may inhibit both K-Ras G12C and K-Ras G12D. In some embodiments, a crystalline compound may inhibit both K-Ras G12V and K-Ras G12C. In some embodiments, a crystalline compound may inhibit both K-Ras G12V and K-Ras G12S. In some embodiments, a compound may inhibit both K-Ras G12D and K-Ras G12V. In some embodiments, a crystalline compound of the present invention inhibits Ras^{WT} in addition to one or more additional Ras mutations (e.g., K-, H- or N-Ras^{WT} and K-Ras G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or N-Ras^{WT} and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{WT} and N-Ras Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T). In some embodiments, a crystalline compound of the present invention inhibits Ras^{amp} in addition to one or more additional Ras mutations (e.g., K-, H- or N-Ras^{amp} and K-Ras G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or N-Ras^{amp} and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{amp} and N-Ras Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T).

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3:145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

In some embodiments, the cancer is non-small cell lung cancer, and the Ras mutation comprises a K-Ras mutation, such as K-Ras G12C, K-Ras G12V or K-Ras G12D. In some embodiments, the cancer is colorectal cancer, and the Ras mutation comprises a K-Ras mutation, such as K-Ras G12C, K-Ras G12V or K-Ras G12D. In some embodiments, the cancer is pancreatic cancer, and the Ras mutation comprises a K-Ras mutation, such as K-Ras G12D or K-Ras G12V. In some embodiments, the cancer is pancreatic cancer, and the Ras mutation comprises an N-Ras mutation, such as N-Ras G12D. In some embodiments, the cancer is melanoma, and the Ras mutation comprises an N-Ras mutation, such as N-Ras Q61R or N-Ras Q61K. In some embodiments, the cancer is non-small cell lung cancer, and the Ras protein is K-Ras^{amp}. In any of the foregoing if not already specified, a compound may inhibit Ras^{WT} (e.g., K-, H- or N-Ras^{WT}) or Ras^{amp} (e.g., K-, H- or N-Ras^{amp}) as well.

In some embodiments, a cancer comprises a Ras mutation and an STK11^{LOF}, a KEAP1, an EPHA5 or an NF1 mutation. In some embodiments, the cancer is non-small cell lung cancer and comprises a K-Ras G12C mutation. In some embodiments, the cancer is non-small cell lung cancer and comprises a K-Ras G12C mutation and an STK11^{LOF} mutation. In some embodiments, the cancer is non-small cell lung cancer and comprises a K-Ras G12C mutation and an STK11^{LOF} mutation. In some embodiments, a cancer comprises a K-Ras G13C Ras mutation and an STK11^{LOF}, a KEAP1, an EPHA5 or an NF1 mutation. In some embodiments, the cancer is non-small cell lung cancer and comprises a K-Ras G12D mutation. In some embodiments, the cancer is non-small cell lung cancer and comprises a K-Ras G12V mutation. In some embodiments, the cancer is colorectal cancer and comprises a K-Ras G12D or G12V mutation. In some embodiments, the cancer is pancreatic cancer and comprises a K-Ras G12D or K-Ras G12V mutation. In some embodiments, the cancer is pancreatic cancer and comprises a K-Ras G12D mutation. In some embodiments, the cancer is pancreatic cancer and comprises a K-Ras G12V mutation. In some embodiments, the cancer is endometrial cancer, ovarian cancer, cholangiocarcinoma, or mucinous appendiceal cancer and comprises a K-Ras G12D or G12V mutation. In some embodiments, the cancer is endometrial cancer, ovarian cancer, cholangiocarcinoma, or mucinous appendiceal cancer and comprises a K-Ras G12D mutation. In some embodiments, the cancer is endometrial cancer, ovarian cancer, cholangiocarcinoma, or mucinous appendiceal cancer and comprises a K-Ras G12V mutation. In some embodiments, the cancer is gastric cancer and comprises a K-Ras G12D or G12V mutation. In some embodiments, the cancer is gastric cancer and comprises a K-Ras G12D mutation. In some embodiments, the cancer is gastric cancer and comprises a K-Ras G12V mutation. In any of the foregoing, a compound may inhibit Ras^{WT} (e.g., K-, H- or N-Ras^{WT}) or Ras^{amp} (e.g., K-, H- or N-Ras^{amp}) as well.

Also provided is a method of inhibiting a Ras protein in a cell, the method comprising contacting the cell with an effective amount of a crystalline compound of the present invention. A method of inhibiting RAF-Ras binding, the method comprising contacting the cell with an effective amount of a crystalline compound of the present invention is also provided. The cell may be a cancer cell. The cancer cell may be of any type of cancer described herein. The cell may be in vivo or in vitro.

### Combination Therapy

The methods of the invention may include a crystalline or salt form of the compound of the invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65: S3-S6 (2005)).

A crystalline form of a compound of the present invention may be administered before, after, or concurrently with one or more of such additional therapies. When combined, dosages of a crystalline compound of the invention and dosages of the one or more additional therapies (e.g., non-drug treatment or therapeutic agent) provide a therapeutic effect (e.g., synergistic or additive therapeutic effect). A crystalline compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the crystalline compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this Combination Therapy section, all references are incorporated by reference for the agents described, whether explicitly stated as such or not.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy. In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a crystalline compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith. A crystalline compound of the present invention may be combined with a second, third, or fourth therapeutic agent, or more. A crystalline compound of the present invention may be combined with one or more therapeutic agents along with one or more non-drug therapies.

For example, a therapeutic agent may be a steroid. Steroids are known in the art. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a crystalline compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. Biologics are known in the art. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. Such checkpoint inhibitors are known in the art. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab). Other anti-TIGIT antibodies are known in the art.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents. Such agents are known in the art.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18: 233a (1999), and Douillard et al., Lancet 355 (9209): 1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammalI and calicheamicin omegalI (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4 (5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, belinostat, LBH 589, romidepsin, ACY-1215, and panobinostat), mTOR inhibitors (e.g., vistusertib, temsirolimus, everolimus, ridaforolimus, and sirolimus), KSP (Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. HER2 inhibitors are known in the art. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. ALK inhibitors are known in the art. Non-limiting examples of ALK inhibitors include ceritinib, TAE-684 (NVP-TAE694), PF02341066 (crizotinib or 1066), alectinib; brigatinib; entrectinib; ensartinib (X-396); Iorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, ERAS-601, SH3809, PF-07284892, or BBP-398), or a pharmaceutically acceptable salt, solvate, isomer (e.g., stereoisomer), prodrug, or tautomer thereof), a SOS1 inhibitor (e.g., BI-1701963, BI-3406, SDR5, BAY-293, MRTX-0902, or RMC-5845, or a pharmaceutically acceptable salt, solvate, isomer (e.g., stereoisomer), prodrug, or tautomer thereof), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, an anti-cancer agent is a SOS1 inhibitor. SOS1 inhibitors are known in the art. In some embodiments, the SOS1 inhibitor is selected from those disclosed in WO 2022219035, WO 2022214594, WO 2022199670, WO 2022146698, WO 2022081912, WO 2022058344, WO 2022026465, WO 2022017519, WO 2021173524, WO 2021130731, WO 2021127429, WO 2021092115, WO 2021105960, WO 2021074227, WO 2020180768, WO 2020180770, WO 2020173935, WO 2020146470, WO 2019201848, WO 2019122129, WO 2018172250, and WO 2018115380, or a pharmaceutically acceptable salt, solvate, isomer (e.g., stereoisomer), prodrug, or tautomer thereof. In some embodiments, a crystalline compound of the present invention is used in combination with a SOS1 inhibitor to treat a K-Ras G13C cancer.

In some embodiments, an anti-cancer agent is an additional Ras inhibitor or a Ras vaccine, or another therapeutic modality designed to directly or indirectly decrease the oncogenic activity of Ras. Such agents are known in the art. In some embodiments, an anti-cancer agent is an additional Ras inhibitor. In some embodiments, the Ras inhibitor targets Ras in its active, or GTP-bound state. In some embodiments, the Ras inhibitor targets Ras in its inactive, or GDP-bound state. In some embodiments, the Ras inhibitor is an inhibitor of K-Ras G12C, such as AMG 510, MRTX1257, MRTX849, JNJ-74699157, LY3499446, ARS-1620, ARS-853, BPI-421286, LY3537982, JDQ443, JAB-3312, JAB-21822, JAB-21000, IBI351, ERAS-3490, RMC-6291, BI 1823911, D-1553, D3S-001, HBI-2438, HS-10370, MK-1084, YL-15293, BBO-8520 (ON/OFF inhibitor), FMC-376 (ON/OFF inhibitor), GEC255, or GDC-6036. In some embodiments, the Ras inhibitor is an inhibitor of K-Ras G12D, such as MRTX1133, JAB-22000, MRTX282, ERAS-4, ERAS-5024, HRS-4642, BI-2852, ASP3082, TH-Z827, TH-7835, RMC-9805, GFH375 (VS-7375), INCB161734 and KD-8. In some embodiments, the Ras inhibitor is a K-Ras G12V inhibitor, such as JAB-23000. In some embodiments, the KRAS (OFF) inhibitor is a pan-KRAS (OFF) inhibitor. In specific embodiments, the pan-KRAS (OFF) inhibitor is JAB-23400, JAB-23425, BI-2493, BI-2865, QTX-3034 (G12D preferring), QTX3544 (G12V preferring), ZG2001, BBO-a, BBO-B, or Pan KRas-IN-1. In some embodiments, the Ras inhibitor is JAB-23400. In some embodiments, the Ras inhibitor is LUNA18. In some embodiments, the Ras inhibitor is BI-2493. In some embodiments, the Ras inhibitor is a K-Ras G12V inhibitor, such as JAB-23000. In some embodiments, the Ras inhibitor is selected from a Ras (ON) inhibitor (that is, Ras in its GTP-bound state) disclosed in the following, incorporated herein by reference in their entireties, or a pharmaceutically acceptable salt, solvate, isomer (e.g., stereoisomer), prodrug, or tautomer thereof: WO 2022235870, WO 2022235864, WO 2021091982, WO 2021091967, WO 2021091956, and WO 2020132597. Other examples of Ras inhibitors are known in the art, such as in the following, incorporated herein by reference in their entireties: WO 2023287896, WO 2023287730, WO 2023284881, WO 2023284730, WO 2023284537, WO 2023283933, WO 2023283213, WO 2023280960, WO 2023280280, WO 2023280136, WO 2023280026, WO 2023278600, WO 2023274383, WO 2023274324, WO 2023034290, WO 2023020523, WO 2023020521, WO 2023020519, WO 2023020518, WO 2023018812, WO 2023018810, WO 2023018809, WO 2023018699, WO 2023015559, WO 2023014979, WO 2023014006, WO 2023010121, WO 2023009716, WO 2023009572, WO 2023004102, WO 2023003417, WO 2023001141, WO 2023001123, WO 2022271923, WO 2022271823, WO 2022271810, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223.

In some embodiments, a therapeutic agent that may be combined with a crystalline compound of the present invention is an inhibitor of the MAP kinase (MAPK) pathway (or “MAPK inhibitor”). Such agents are known in the art. MAPK inhibitors include, but are not limited to, one or more MAPK inhibitor described in Cancers (Basel) 2015 September; 7 (3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLOS One. 2014 Nov. 25; 9 (11)); and GSK1120212 (or JTP-74057, described in Clin Cancer Res. 2011 Mar. 1; 17 (5): 989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. Such agents are known in the art. The PI3K/AKT inhibitor may include, but is not limited to, one or more PI3K/AKT inhibitor described in Cancers (Basel) 2015 September; 7 (3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist. Such agents are known in the art.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, additional therapeutic agents include FGFR inhibitors, PARP inhibitors, BET inhibitors, PRMT5i inhibitors, MAT2A inhibitors, VEGF inhibitors, and HDAC inhibitors. In some embodiments, a therapeutic agent may be a pan-RTK inhibitor, such as afatinib.

IGF-1R inhibitors are known in the art and include linsitinib, or a pharmaceutically acceptable salt thereof.

EGFR inhibitors are known in the art and include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15: 59 (8): 1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics in Oncology Therapeutic Antibody Development, BioTechniques 2005, 39 (4): 565-8; and Paez et al., EGFR Mutations in Lung Cancer Correlation with Clinical Response to Gefitinib Therapy, Science 2004, 304 (5676): 1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8 (12): 1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

MEK inhibitors are known in the art and include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

PI3K inhibitors are known in the art and include, but are not limited to, wortmannin; 17-hydroxywortmannin analogs described in WO06/044453; 4-[2-(1H-Indazol-4-yl)-6-[[4-(methylsulfonyl) piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-I-(4-((2-(2-aminopyrimidin-5-yl)-7-methyl-4-morpholinothieno[3,2-d]pyrimidin-6-yl)methyl) piperazin-1-yl)-2-hydroxypropan-1-one (described in WO08/070740); LY294002 (2-(4-morpholinyl)-8-phenyl-4H-I-benzopyran-4-one (available from Axon Medchem); PI 103 hydrochloride (3-[4-(4-morpholinylpyrido-[3′,2′: 4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrimidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors are known in the art and include, but are not limited to, Akt-1-1 (inhibits Aktl) (Barnett et al., Biochem. J. 2005, 385 (Pt. 2): 399-408); Akt-1-1,2 (inhibits Akl and 2) (Barnett et al., Biochem. J. 2005, 385 (Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134 (12 Suppl): 3493S-3498S); perifosine (e.g., interferes with Akt membrane localization; Dasmahapatra et al. Clin. Cancer Res. 2004, 10 (15): 5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors are known in the art and include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl) rapamycin; 40-[3-hydroxy (hydroxymethyl)methylpropanoate]-rapamycin (also known as CC1779); 40-epi-(tetrazolyt)-rapamycin (also called ABT578); 32-deoxorapamycin; 16-pentynyloxy-32 (S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552, having the structure

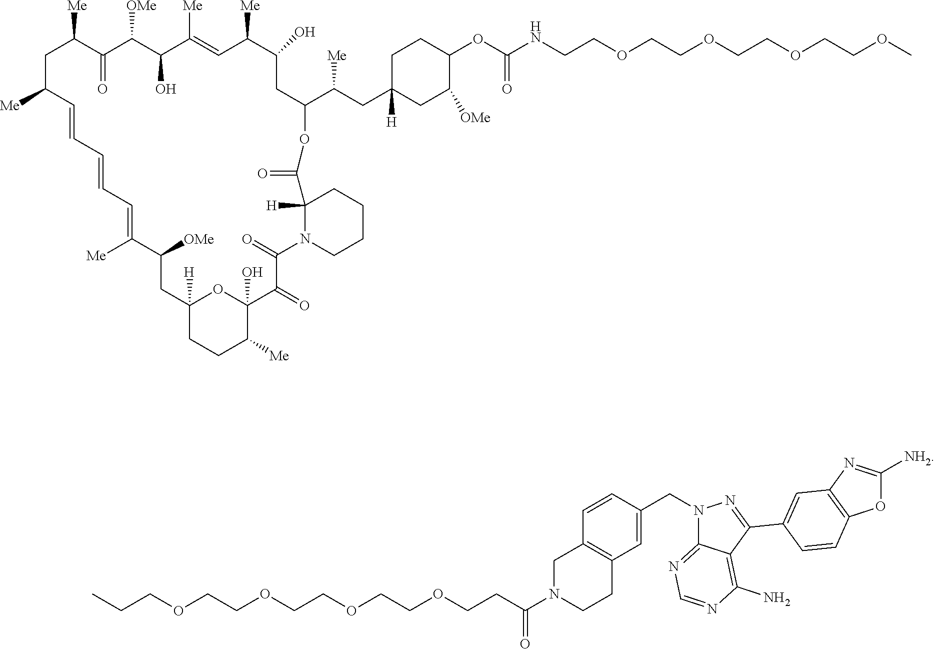

BRAF inhibitors that may be used in combination with compounds of the invention are known in the art and include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E.

MCL-1 inhibitors are known in the art and include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 inhibitors are known in the art. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung, and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and patent applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2021176072, WO 2021171261, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Pat. Nos. 11,179,397, 11,044,675, 11,034,705, 11,033,547, 11,001,561, 10,988,466, 10,954,243, 10,934,302, or 10,858,359, or a pharmaceutically acceptable salt, solvate, isomer (e.g., stereoisomer), prodrug, or tautomer thereof, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155, having the structure:

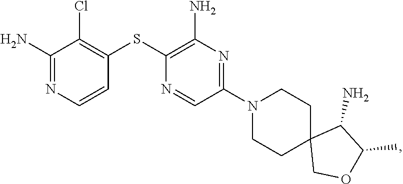 [SMILES: C[C@@H]1OCC2(CCN(c3cnc(Sc4ccnc(N)c4Cl)c(N)n3)CC2)[C@@H]1N]

or a pharmaceutically acceptable salt, solvate, isomer (e.g., stereoisomer), prodrug, or tautomer thereof. In some embodiments, the SHP2 inhibitor is RMC-4550. In some embodiments, the SHP2 inhibitor is RMC-4630, having the structure:

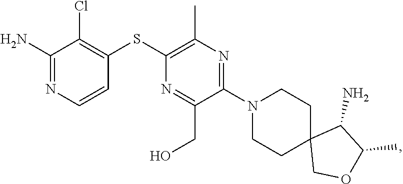 [SMILES: Cc1nc(N2CCC3(CC2)CO[C@@H](C)[C@H]3N)c(CO)nc1Sc1ccnc(N)c1Cl]

or a pharmaceutically acceptable salt, solvate, isomer (e.g., stereoisomer), prodrug, or tautomer thereof. In some embodiments, the SHP2 inhibitor is JAB-3068, having the structure

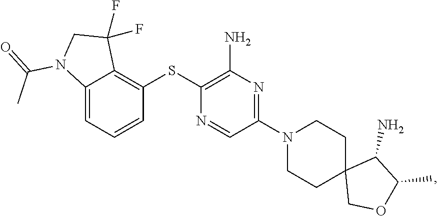 [SMILES: CC(=O)N1CC(F)(F)c2c(Sc3ncc(N4CCC5(CC4)CO[C@@H](C)[C@H]5N)nc3N)cccc21]

or a pharmaceutically acceptable salt, solvate, isomer (e.g., stereoisomer), prodrug, or tautomer thereof. In some embodiments, the SHP2 inhibitor is JAB-3312. In some embodiments, the SHP2 inhibitor is the following compound,

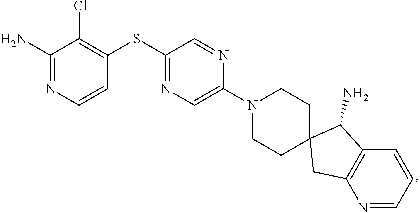 [SMILES: Nc1nccc(Sc2cnc(N3CCC4(CC3)Cc3ncccc3[C@H]4N)cn2)c1Cl]

or a pharmaceutically acceptable salt, solvate, isomer (e.g., stereoisomer), prodrug, or tautomer thereof. In some embodiments, the SHP2 inhibitor is RLY-1971, having the structure

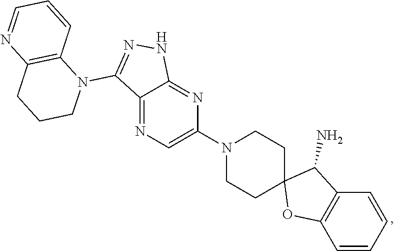 [SMILES: N[C@@H]1c2ccccc2OC12CCN(c1cnc3c(N4CCCc5ncccc54)n[nH]c3n1)CC2]

or a pharmaceutically acceptable salt, solvate, isomer (e.g., stereoisomer), prodrug, or tautomer thereof. In some embodiments, the SHP2 inhibitor is ERAS-601. In some embodiments, the SHP2 inhibitor is BBP-398.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a Ras inhibitor of the present invention is used in combination with a MEK inhibitor and a SOS1 inhibitor. In some embodiments, a Ras inhibitor of the present invention is used in combination with a PD-L1 inhibitor and a SOS1 inhibitor. In some embodiments, a Ras inhibitor of the present invention is used in combination with a PD-L1 inhibitor and a SHP2 inhibitor. In some embodiments, a Ras inhibitor of the present invention is used in combination with a MEK inhibitor and a SHP2 inhibitor. In some embodiments, a Ras inhibitor of the present invention is used in combination with a SHP2 inhibitor and a Ras inhibitor that inhibits multiple Ras isoforms and/or mutants. In some embodiments, the cancer is lung cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, the cancer is colorectal cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, a Ras inhibitor of the present invention is used in combination with an immunotherapy, optionally in combination with a chemotherapeutic agent.

Proteasome inhibitors are known in the art and include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents). Other immune therapies are known in the art.

Immunomodulatory agents (IMiDs) are a class of immunomodulatory drugs (drugs that adjust immune responses) containing an imide group. The IMID class includes thalidomide and its analogues (lenalidomide, pomalidomide, and apremilast).

Exemplary anti-PD-1 antibodies and methods for their use are described by Goldberg et al., Blood 2007, 110 (1): 186-192; Thompson et al., Clin. Cancer Res. 2007, 13 (6): 1757-1761; and WO06/121168 A1), as well as described elsewhere herein.

FGFR inhibitors are known in the art, such as pemigatinib and erdafitinib, including FGFR2 inhibitors and FGFR4 inhibitors. See, e.g., Cancers (Basel), 2021 June; 13 (12) 2968.

BET inhibitors are known in the art, such as romidepsin, panobinostat and belinostat. See, e.g., British J. Cancer 124:1478 (2021).

PRMT5i inhibitors are known in the art, such as PF-0693999, PJ-68 and MRTX1719. See, e.g., Biomed. Pharmacotherapy 144:112252 (2021).

MAT2A inhibitors are known in the art, such as AG-270 and IDE397. See, e.g., Exp Opin Ther Patents (2022) DOI: 10.1080/13543776.2022.2119127.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the crystalline compounds of the invention is an anti-angiogenic agent. Anti-angiogenic agents are known in the art and are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), VEGF inhibitors, EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXIGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-Ialfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met. Such agents are known in the art.

Another example of a therapeutic agent that may be used in combination with compounds of the invention is an autophagy inhibitor. Autophagy inhibitors are known in the art and include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of CAMP, and drugs which elevate CAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with the crystalline compounds of the invention is an anti-neoplastic agent, which are known in the art. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-Ia, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with crystalline compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The crystalline compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a crystalline compound described herein and any of the agents described herein can be formulated together in the same dosage form and administered simultaneously. Alternatively, a crystalline compound of the invention and any of the therapies described herein can be simultaneously administered, wherein both the agents are present in separate formulations. In another alternative, a crystalline compound of the present disclosure can be administered and followed by any of the therapies described herein, or vice versa. In some embodiments of the separate administration protocol, a crystalline compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a crystalline compound of the invention) described herein, and (b) a package insert with instructions to perform any of the methods described herein. In some embodiments, the kit includes (a) a pharmaceutical composition including an agent (e.g., a crystalline compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a crystalline compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The following examples further illustrate the invention but, of course, should not be construed as in any way limiting its scope.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1

This example demonstrates exemplary methods of preparing the crystalline Form 1 of Compound A free base in accordance with an embodiment of the invention.

In one method, about 50 mg of amorphous Compound A free base was dissolved in the minimal amount of MeOH at ambient temperature (about 20-25° C.). To the resulting clear solution, a 4-fold volume of water was quickly added, which formed precipitate. The resulting precipitate was isolated by centrifugation through a 0.45-μm nylon membrane filter at 14,000 rpm to obtain crystalline Form 1 of Compound A free base.

In another method, 1 mL of a saturated solution of Compound A in water at 50° C. was added to 1 mL of methanol. The hazy solution was allowed to cool to ambient temperature to obtain crystalline Form 1 of Compound A free base. Crystals that appeared to be of sufficient size and quality for single-crystal X-ray diffraction studies were removed and characterized by a crystallographer.

In another method, a mixture of 201.3 mg of amorphous Compound A and 2.5 mL of an aqueous solution of acetate buffer (pH 4) was stirred at ambient temperature for about 7 days. The mixture was centrifuged, the liquid was decanted, and the remaining solid was allowed to dry in the air to obtain crystalline Form 1 of Compound A free base.

In another method, about 50 mg of amorphous Compound A was dissolved in a minimal amount of acetone to form a clear solution at ambient temperature (e.g., about 20-25° C.). To this solution, water was added until a large amount of solids precipitated (about 1:1 acetone/water v/v). The solids were collected by centrifugation through a 0.45 μm nylon membrane filter at 14,000 rpm in order to obtain crystalline Form 1.

In another method, 9.5 g of amorphous Compound A was added to 38 mL of MeOH. The mixture was filtered to obtain a clear solution. The clear solution was quickly added to 152 mL of water to form a suspension. To the suspension, seeds of Form 1 were added, and the mixture was stirred at 25° C. for 24 h, then stirred at 5° C. for 24 h. Solids were collected by vacuum filtration. After drying the solids at 25° C. for about six days, 8.2 g of Form 1 was obtained as a white powder (78% yield).

In another method, the crystalline Form 5 of Compound A (see Example 13) spontaneously converts to crystalline Form 1 of Compound A when crystalline Form 5 is exposed to water activity≥0.4 at 25° C.

### Example 2

This example demonstrates X-Ray Powder Diffraction (XRPD) characterization of the crystalline Form 1 of Compound A free base in accordance with an embodiment of the invention.

XRPD measurements were carried out using one of two Rigaku Smart-Lab X-ray diffraction systems. Each was configured for Bragg-Brentano reflection geometry using a line source X-ray beam. The Bragg-Brentano geometry was controlled by passive divergence and receiving slits with the sample itself acting as the focusing component for the optics. Powder samples were prepared in low-background Si holders using light manual pressure to keep the sample surfaces flat and level with the reference surface of the sample holder. Diffractometer specifications and data collection parameters for diffraction system 1 are shown in Table 1. Diffractometer specifications and data collection parameters for diffraction system 2 are shown in Table 2. The XRPD of crystalline Form 1 of Compound A as the mixed methanol is shown in FIG. 1A. In an essentially pure material of crystalline Form 1 as the mixed methanol and water solvate, peaks can be observed at angles of refraction 20 as set forth in Tables 3 and 4 (runs 1 and 2, respectively). The XRPD of crystalline Form 1 as the tri-hydrate solvate is shown in FIG. 1B. In an essentially pure material of crystalline Form 1 as the tri-hydrate solvate, peaks can be observed at angles of refraction 20 as set forth in Table 5.

TABLE 1
Diffractometer System 1 Specifications
and Data Collection Parameters.

| Parameter | Value |
|---|---|
| Geometry | Reflection |
| Tube Anode | Cu Kα1 |
| Tube Type | Long Fine Focus |
| Tube Voltage (kV) | 40 |
| Tube Current (mA) | 44 |
| Detector | D/teX Ultra PSD^{a} |
| Monochromatization | Kβ Filter |
| Incident Slit (°) | ⅓ |
| Receiving Slit 1 (mm) | 18 |
| Receiving Slit 2 (mm) | open |
| Start Angle (°2θ) | 2 |
| End Angle (°2θ) | 40 |
| Step Size (°2θ) | 0.02 |
| Scan Speed (°2θ/min) | 6 |
| Spinning (rpm) | 11 |
| Sample Holder | low-background Si |

^{a}PSD = position sensitive detector

TABLE 2
Diffractometer System 2 Specifications
and Data Collection Parameters.

| Parameter | Value |
|---|---|
| Geometry | Reflection |
| Tube Anode | Cu Kα1 |
| Tube Type | Long Fine Focus |
| Tube Voltage (kV) | 40 |
| Tube Current (mA) | 44 |
| Detector | HyPix3000 |
| Monochromatization | Focusing mirror |
| Incident Slit (°) | ⅓ |
| Receiving Slit 1 (mm) | 18 |
| Receiving Slit 2 (mm) | 20 |
| Start Angle (°2θ) | 2 |
| End Angle (°2θ) | 40 |
| Step Size (°2θ) | 0.02 |
| Scan Speed (°2θ/min) | 6 |
| Spinning (rpm) | 11 |
| Sample Holder | low-background Si |

TABLE 3
X-ray powder diffraction peaks of crystalline Form 1 of Compound
A free base as the mixed methanol and water solvate (run 1).

| Position [2θ°] | Relative Intensity |
|---|---|
| 5.2 | 77 |
| 6.9 | 9 |
| 8.4 | 18 |
| 8.9 | 35 |
| 9.2 | 23 |
| 10.0 | 34 |
| 10.3 | 37 |
| 10.5 | 32 |
| 10.7 | 43 |
| 11.7 | 25 |
| 12.2 | 29 |
| 12.5 | 20 |
| 13.2 | 21 |
| 13.6 | 9 |
| 14.4 | 84 |
| 14.8 | 51 |
| 15.1 | 19 |
| 15.5 | 29 |
| 16.3 | 59 |
| 16.7 | 71 |
| 17.0 | 100 |
| 17.3 | 50 |
| 17.6 | 22 |
| 18.2 | 41 |
| 18.4 | 59 |
| 18.9 | 33 |
| 19.1 | 38 |
| 20.2 | 31 |
| 20.5 | 60 |
| 20.6 | 45 |
| 20.9 | 44 |
| 21.2 | 20 |
| 21.6 | 28 |
| 21.9 | 21 |
| 22.6 | 16 |
| 23.1 | 16 |
| 23.5 | 30 |
| 23.7 | 32 |
| 24.0 | 37 |
| 24.5 | 29 |
| 24.8 | 19 |
| 25.1 | 28 |
| 25.7 | 25 |
| 26.3 | 17 |
| 26.9 | 16 |
| 27.3 | 22 |
| 27.5 | 14 |
| 28.0 | 11 |
| 28.8 | 13 |
| 30.2 | 17 |
| 30.9 | 16 |
| 30.9 | 16 |
| 31.5 | 11 |
| 32.0 | 11 |
| 32.6 | 9 |
| 32.9 | 11 |
| 33.6 | 12 |
| 35.5 | 11 |
| 36.2 | 11 |
| 36.7 | 10 |
| 37.5 | 9 |
| 38.4 | 8 |
| 39.5 | 10 |

TABLE 4
X-ray powder diffraction peaks of crystalline Form 1 of Compound
A free base as the mixed methanol and water solvate (run 2).

| Position [2θ°] | Relative Intensity |
|---|---|
| 3.5 | 2.10 |
| 5.2 | 46.60 |
| 6.8 | 3.30 |
| 8.4 | 9.60 |
| 8.6 | 26.60 |
| 9.1 | 12.30 |
| 9.9 | 17.50 |
| 10.3 | 24.40 |
| 10.6 | 27.40 |
| 11.6 | 18.60 |
| 11.9 | 3.50 |
| 12.2 | 29.00 |
| 12.4 | 16.10 |
| 13.2 | 10.00 |
| 13.5 | 5.70 |
| 14.3 | 100.00 |
| 14.6 | 18.50 |
| 14.7 | 30.00 |
| 15.0 | 10.10 |
| 15.4 | 23.40 |
| 16.3 | 33.50 |
| 16.8 | 68.40 |
| 17.0 | 49.20 |
| 17.1 | 30.40 |
| 17.5 | 19.20 |
| 18.2 | 31.80 |
| 18.4 | 13.90 |
| 18.8 | 15.30 |
| 19.0 | 27.80 |
| 19.9 | 2.10 |
| 20.3 | 35.30 |
| 20.5 | 49.70 |
| 20.8 | 24.30 |
| 21.0 | 21.50 |
| 21.5 | 11.20 |
| 21.8 | 7.70 |
| 22.3 | 5.60 |
| 22.6 | 7.80 |
| 23.0 | 3.90 |
| 23.3 | 22.60 |
| 23.6 | 7.40 |
| 23.7 | 16.00 |
| 24.0 | 30.00 |
| 24.4 | 21.20 |
| 24.8 | 9.00 |
| 25.1 | 18.60 |
| 25.8 | 19.00 |
| 26.3 | 4.00 |
| 26.8 | 3.80 |
| 27.1 | 13.80 |
| 27.5 | 4.00 |
| 27.7 | 4.40 |
| 28.9 | 4.80 |
| 29.4 | 3.70 |
| 29.8 | 6.00 |
| 30.2 | 4.20 |
| 30.7 | 7.80 |
| 31.1 | 4.10 |
| 31.3 | 4.10 |
| 31.8 | 3.00 |
| 32.0 | 2.90 |
| 32.4 | 2.40 |
| 32.9 | 2.50 |
| 33.7 | 4.30 |
| 35.6 | 3.40 |
| 36.1 | 2.50 |
| 36.9 | 2.80 |
| 39.3 | 3.00 |

TABLE 5
X-ray powder diffraction peaks of crystalline Form 1
of Compound A free base as the tri-hydrate solvate.

| Position [2θ°] | Relative Intensity |
|---|---|
| 5.2 | 88 |
| 6.8 | 11 |
| 8.4 | 20 |
| 8.6 | 36 |
| 9.2 | 28 |
| 10.0 | 32 |
| 10.3 | 37 |
| 10.6 | 48 |
| 11.6 | 28 |
| 12.2 | 41 |
| 12.4 | 21 |
| 13.2 | 23 |
| 14.3 | 92 |
| 14.8 | 45 |
| 15.1 | 20 |
| 15.4 | 36 |
| 16.3 | 59 |
| 16.8 | 87 |
| 16.9 | 95 |
| 16.9 | 100 |
| 17.2 | 53 |
| 17.5 | 24 |
| 18.1 | 43 |
| 18.3 | 61 |
| 18.4 | 34 |
| 18.8 | 37 |
| 19.0 | 40 |
| 20.4 | 75 |
| 20.8 | 41 |
| 21.5 | 30 |
| 21.8 | 21 |
| 22.2 | 16 |
| 22.6 | 20 |
| 23.3 | 35 |
| 23.7 | 34 |
| 23.9 | 43 |
| 24.0 | 43 |
| 24.4 | 34 |
| 24.7 | 24 |
| 25.1 | 33 |
| 25.4 | 16 |
| 25.8 | 34 |
| 26.3 | 16 |
| 26.8 | 17 |
| 27.1 | 25 |
| 28.8 | 14 |
| 29.4 | 13 |
| 29.8 | 15 |
| 30.1 | 16 |
| 30.7 | 16 |
| 31.1 | 13 |
| 31.9 | 11 |
| 32.4 | 9 |
| 32.8 | 12 |
| 33.6 | 13 |
| 36.8 | 11 |
| 37.6 | 9 |
| 38.3 | 9 |
| 39.3 | 10 |

### Example 3

This example demonstrates single crystal X-ray crystallography characterization of the crystalline Form 1 of Compound A free base in accordance with an embodiment of the invention. The X-ray crystal structure of crystalline Form 1 of Compound A free base (asymmetric unit) is shown in FIG. 2. The structure was determined to be Compound A free base as a mixed methanol and water solvate.

A portion of Compound A free base was selected and a light brown, plate-shaped crystal with formula 2 (C₄₄H₅₈N₈O₅S)·5.55(H₂O)·2.19(CH₄O), having approximate dimensions of 0.42× 0.19× 0.04 mm, was mounted on a Mitegen micromesh mount in a random orientation. Preliminary examination and data collection were performed using Cu Kα radiation (λ=1.54178 Å) on Bruker AXS D8 Quest CMOS diffractometer equipped with a four-axis kappa stage, an I-μ-S microsource X-ray tube, laterally-graded multilayer optics, a PhotonIII-C14 single photon counting detector, and an Oxford Cryosystems low-temperature device. The initial unit cell was determined and data were collected using Apex3 v2019.11-03 at a temperature of 150 K. Frames were integrated using SAINT V8.40B.³ A total of 59, 167 reflections were collected, of which 19,822 were unique. Cell constants for data collection were obtained from least-squares refinement using 9,955 reflections between 4.1903 and 78.2402°. The triclinic cell parameters and calculated volume are a=10.8182 (3) Å, b=13.3787 (4) Å, c=17.6881 (5) Å, α=88.4427 (15)°, β=79.5367 (16)°, γ=82.0377 (16°) and V=2493.21 (12) Å³. For Z=1 and a formula weight of 1792.50 the calculated density is 1.194 g/cm³. The linear absorption coefficient is 1.055/mm for Cu Kα radiation. Scaling and a multi-scan absorption correction using SADABS 2016-24 was applied. Transmission coefficients ranged from 0.6221 to 0.7543. Intensities of equivalent reflections were not averaged during data processing.

The space group was determined by the program XPREP as embedded in SHELXTL.⁵ Intensity statistics indicated the space group P1 (#1). The structure was solved by direct methods using SHELXS^{6a} and refined by full matrix least squares against F² with all reflections using SHELXL-2018⁶ and the graphical user interface ShelXle⁷. Additional atoms were located in succeeding difference Fourier syntheses. The structure was refined using full-matrix least-squares where the function minimized was Σw(|Fₒ|²−|F_{c}|²)² and the weight w is defined as w=1/[σ2 (Fₒ²)+(0.0831P)²+0.3910P] where P=(Fₒ²+2F_{c}²)/3. Scattering factors were taken from the International Tables for Crystallography (Vol C Tables 4.2.6.8 and 6.1.1.4). A total of 19,822 independent reflections were used in the refinements. A total of 7,187 reflections with F2>2 σ (F2) were used in the calculation of R1.

Hydrogen atoms attached to carbon were positioned geometrically and constrained to ride on their parent atoms. Carbon-hydrogen bond distances were constrained to 0.95 Å for aromatic and alkene CH moieties, and to 1.00, 0.99 and 0.98 Å for aliphatic CH, CH2 and CH3 moieties, respectively. Nitrogen-hydrogen bond distances were constrained to 0.88 Å for planar (sp² hybridized) nitrogen atoms. Oxygen-hydrogen distances of alcohols were constrained to 0.84 Å. Hydrogen atoms on methyl and alcohol moieties were initially allowed rotate to best fit the experimental electron density. When necessary, alcohol H-atom positions were further restrained based on hydrogen-bonding considerations. Some H atoms of disordered methyl groups were set to be in staggered positions in the final refinement cycles. Hydrogen atoms of pyramidalized R₂NH groups were refined an N—H distances were retrained to 0.88 (2) Å. Water H-atom positions were initially refined and O—H and H . . . H distances were restrained to 0.84 (2) and 1.36 (2) Å, respectively. When necessary, water H-atom positions were further restrained based on hydrogen bonding considerations. In the final refinement cycles positions of water and alcohol hydrogen atoms were set to ride on those of their carrier O atoms. Uᵢₛₒ(H) values were set to a multiple of U_{eq}(C) with 1.5 for OH and CH₃, and 1.2 for CH and CH₂ units, respectively.

Disorder is observed for some sections of both crystallographically-independent molecules. Additional extensive disorder is observed for interstitial solvate molecules (methanol and water). In molecule A and B, disorder is observed for the methylcyclopropane-carboxamide group. In molecule B, that disorder extends into the main molecule including the thiazole and hexahydropyridazine rings. In molecule B, the piperazine ring is also disordered.

For all disordered segments, equivalent moieties of both molecules were restrained to have similar geometries. Uij components of atomic displacement parameters (ADPs) for disordered atoms closer to each other than 2.0 Å were restrained to be similar. Subject to these conditions the occupancy ratios refined to 0.593 (17) to 0.407 (17) for the methylcyclopropane-carboxamide group in molecule A, 0.612 (2) to 0.388 (2) for the methylcyclopropane-carboxamide/thiazole/hexahydropyridazine disorder in molecule B; and to 0.681 (11) to 0.319 (11) for the piperazine disorder in molecule B.

The oxygen atom of a methanol molecule (O13, C47) was refined as disordered, being hydrogen-bonded to either methoxy O4B, or pyridyl N5B. The carbon atom was excluded from the disorder. The C—O bond distances were restrained to be similar to each other, and to those of two other partially occupied methanol molecules. Uij components of ADPs for disordered atoms closer to each other than 2.0 Å were restrained to be similar. The minor moiety hydroxyl H atom position was restrained based on hydrogen bonding considerations. Subject to these conditions the occupancy ratio refined to 0.789 (13) to 0.211 (13). A site encapsulated by the main molecules was refined as two partially occupied water molecules (O12B, O12D). Uij components of ADPs for the two O atoms were restrained to be similar. Occupancy rates refined to 0.171 (11) and 0.292 (13).

A solvate molecule was refined as disordered between methanol (O2, C1) and water, with the water molecule and one H atom shared between the two molecules. A neighboring water molecule was included in the disorder sharing the occupancy ratio of the methanol/water disorder. The disorder ratio refined to 0.550 (4) to 0.450 (4) in favor of methanol.

Adjacent to this disorder and associated with the methylcyclopropane-carboxamide disorder of both molecules is additional disorder of water and methanol molecules. Disorder is extensive and partially ill resolved. No unambiguous self-consistent disorder model could be developed due to extensive overlap of organic fragments with solvate molecules, ill-resolved disorder within disorder and change between occupancy rates of neighboring solvate molecules. The disorder was approximated using partial occupancies. Where possible, occupancy rates of overlapping moieties were constrained to sum up to unity, but some unresolved difference density that could not be modeled remained.

Two disordered methanol molecules were refined as disordered and partially occupied (O8B, C45B; O8D C45D). The C—O bond distances were restrained to be similar to each other, and to those of the other disordered methanol molecule (see above). Uij components of ADPs for disordered atoms closer to each other than 2.0 Å were restrained to be similar. Occupancy rates refined to 0.251 (11) (O8B) and 0.388 (2) (O8D). Occupancies of disordered water molecules refined to the following values:
O7B: 0.612 (2) (same as major moiety of molecule B methylcyclopropane-carboxamide disorder)
O9B1: 0.450 (4) (same as water occupancy of methanol/water disorder of 02)
O9B2: 0.162 (4) (shares site with O9D)
O10B: 0.489 (17) (partially occupied site)
O10C: 0.388 (19) (same as minor moiety of molecule B methylcyclopropane-carboxamide disorder)
O7D: 0.169 (13) (partially occupied site)
O9D: 0.388 (2) (shares site with O9B2)

The final cycle of refinement included 1,598 variable parameters and 1,445 restraints and converged (the largest parameter shift was 0.007 times its standard uncertainty) with unweighted and weighted agreement factors of:

$$\\ R 1 = \sum \vert {F}_{o} \vert - \vert {F}_{c} \vert / \sum \vert {F}_{o} \vert = 0.0483$$

$$\\ wR 2 = {\{ \sum [ w ( {F}_{o}^{{}_{} 2} - {F}_{c}^{{}_{} 2} ) 2 ] / \sum [ {w ( {F}_{o}^{{}_{} 2} )}^{2} ] \}}^{0.5} = 0.1381$$

The goodness-of-fit parameter was 1.031. The highest peak in the final difference Fourier map had a height of 0.693 e/Å3. The minimum negative peak had a height of −0.271 e/Å3. Crystal data and data collection parameters are given in Table 6.

TABLE 6
Crystal Data and Data Collection and Refinement Parameters.
Crystal Data

| Formula | 2(C₄₄H₅₈N₈O₅S)•5.55(H₂O)•2.19(CH₄O) |
|---|---|
| Formula weight | 1792.50 |
| Crystal system, space | Triclinic, P1 |
| group | |
| Data collection | 150 |
| temperature (K) | |
| a, b, c (Å) | 10.8182(3), 13.3787(4), 17.6881(5) |
| α, β, γ (°) | 88.4427(15), 79.5367(16), 82.0377(16) |
| Volume (Å³) | 2493.21(12) |
| Z | 1 |
| F₀₀₀ | 963 |
| Dₓ (mg m⁻³) | 1.194 |
| Radiation type | Cu Kα |
| No. of reflections for | 9955 |
| cell measurement | |
| Linear absorption | 1.06 |
| coefficient μ (mm⁻¹) | |
| Crystal shape | Plate |
| Crystal color | Light brown |
| Crystal size (mm) | 0.42 × 0.19 × 0.04 |

Data Collection

| | |
|---|---|
| Diffractometer | Bruker AXS D8 Quest diffractometer with |
| | PhotonII charge-integrating pixel array |
| | detector (CPAD) |
| Radiation source | I-mu-S microsource X-ray tube |
| Monochromator | Laterally graded multilayer (Goebel) mirror |
| Detector resolution | 7.4074 |
| Scan method | ω and phi scans |
| Absorption correction | Multi-scan, SADABS 2016/2⁴ |
| Tₘᵢₙ, Tₘₐₓ | 0.622, 0.754 |
| No. of measured, | 59167, 19822, 17187 |
| independent, and | |
| observed [I > 2σ(I)] | |
| reflections | |
| Rᵢₙₜ | 0.039 |
| θₘₐₓ, θₘᵢₙ (°) | 79.8, 4.2 |
| (sin θ/λ)ₘₐₓ (Å⁻¹) | 0.638 |
| range of h, k, l | h = −13→13, k = −16→15, l = −22→22 |

Refinement

| | |
|---|---|
| Refinement on | F² |
| R[F² > 2σ(F²)], wR(F²), S | 0.048, 0.138, 1.03 |
| Number of reflections | 19822 |
| Number of parameters | 1598 |
| Number of restraints | 1445 |
| H-atom treatment | H atoms treated by a mixture of independent |
| | and constrained refinement |
| Weighting scheme | w = 1/[σ²(Fₒ²) + (0.0831P)² + |
| | 0.391P] where P = (Fₒ² + 2F_{c}²)/3 |
| (Δ/σ)ₘₐₓ | 0.007 |
| Δðₘₐₓ, Δðₘᵢₙ (e Å⁻³) | 0.69, −0.27 |
| Absolute structure | Refined as an inversion twin |
| Absolute structure | 0.008(19) |
| parameter | |

### Example 4

This example demonstrates differential scanning calorimetry (DSC) characterization of the crystalline forms of Compound A in accordance with an embodiment of the invention.

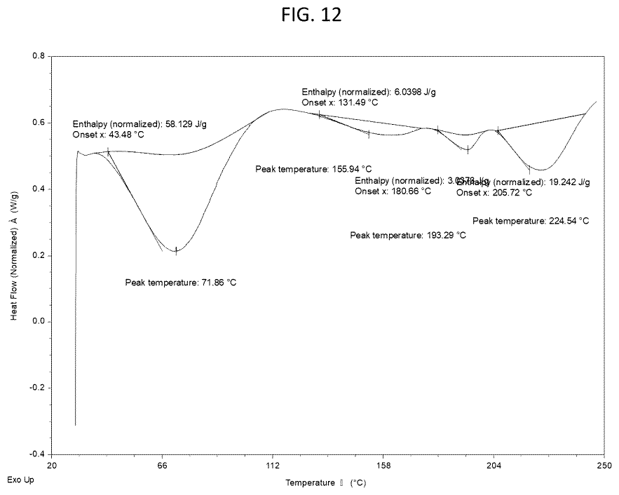

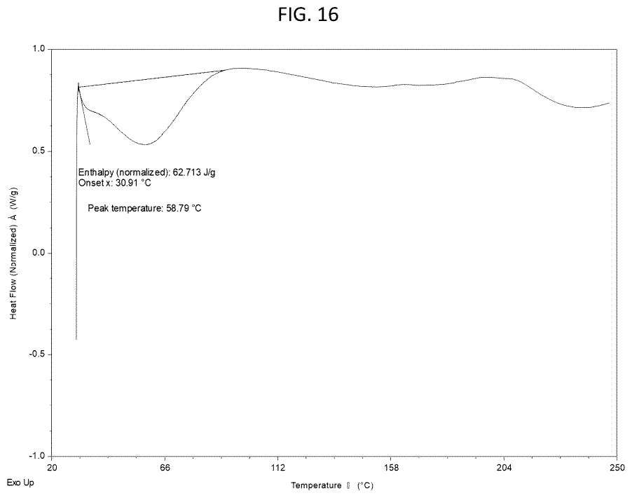

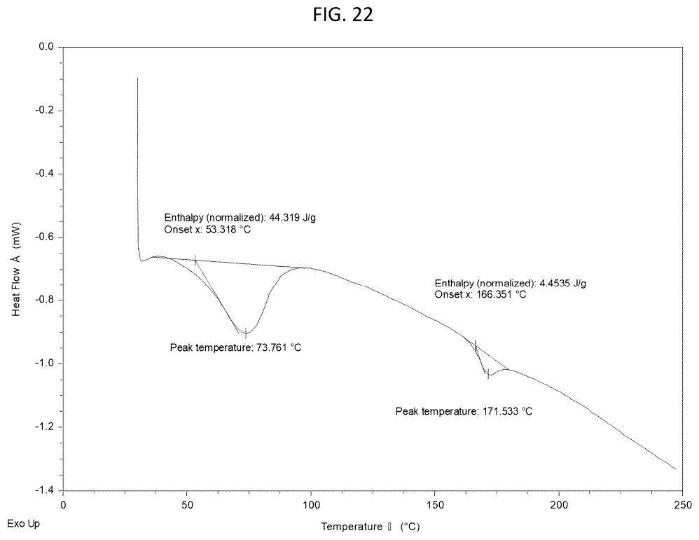

The DSC analysis was carried out using a TA Instruments Q2500 Discovery Series instrument. The instrument temperature calibration was performed using indium. The DSC cell was kept under a nitrogen purge of ˜50 mL per minute during each analysis. The sample was placed in a standard, crimped, aluminum pan and was heated from approximately 25° C. to 350° C. at a rate of 10° C. per minute. The DSC thermogram of crystalline Form 1 of Compound A free base is shown in FIG. 3. The DSC thermogram of crystalline Compound A tosylate salt is shown in FIG. 7. The DSC thermogram of crystalline Form 2 of Compound A is shown in FIG. 12. The DSC thermogram of crystalline Form 4 of Compound A is shown in FIG. 16. The DSC thermogram of crystalline Form 5 of Compound A is shown in FIG. 19. The DSC thermogram of crystalline Form 6 of Compound A is shown in FIG. 22.

### Example 5

This example demonstrates thermogravimetry (TG) characterization of the crystalline forms of Compound A in accordance with an embodiment of the invention.

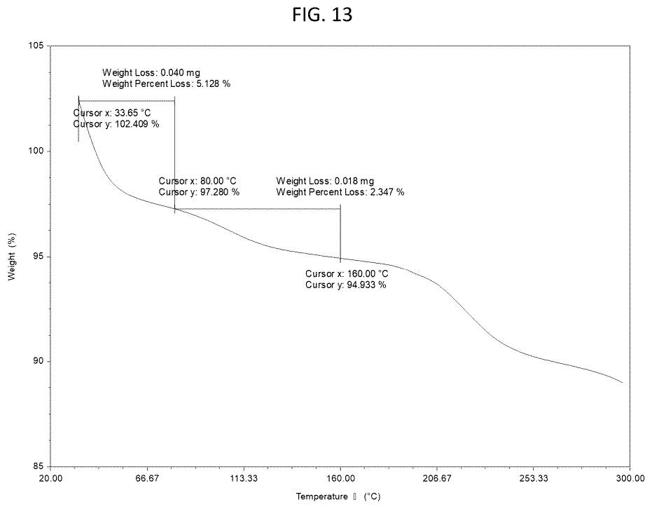

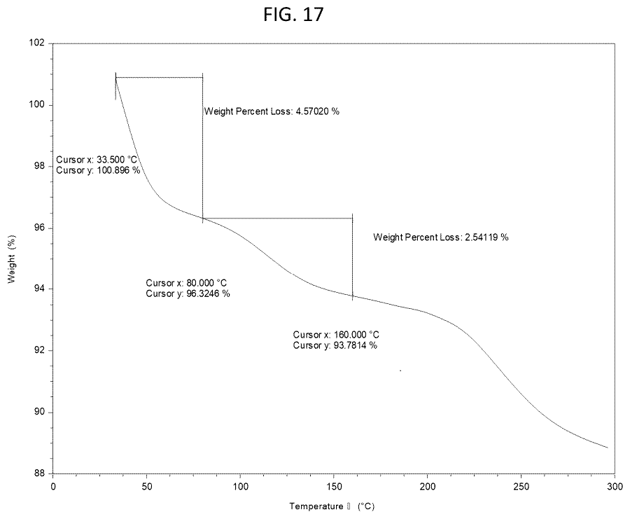

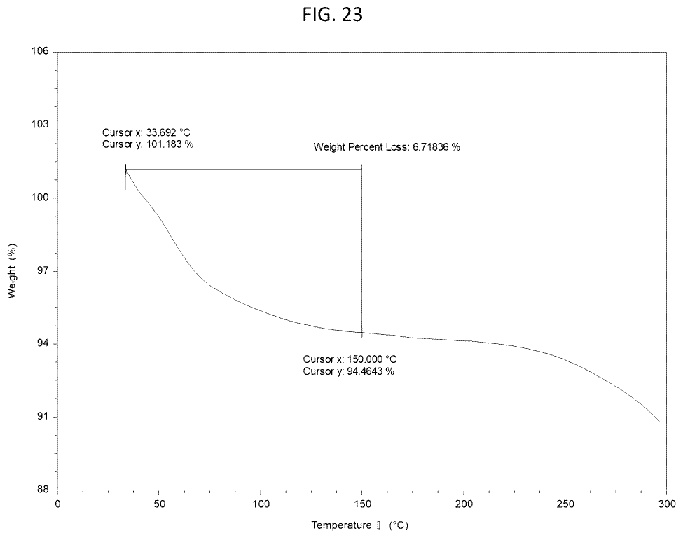

TG analyses were carried out using a TA Instruments Discovery TGA 5500 instrument that was cooled using a TA Instruments Refrigerated Cooling System (RCS) 90 chiller. The instrument balance was calibrated using class M weights and temperature calibration was performed using alumel. Each sample was loaded onto a platinum sample pan and the pan was loaded into the TG instrument. The pan was heated from ambient temperature to 350° C. at a rate of 10° C. per minute. The instrument was controlled using TA Trios software v 5.0.0.44608. The graph of the thermogravimetric analysis (TGA) of crystalline Form 1 of Compound A free base is shown in FIG. 4. The graph of the thermogravimetric analysis (TGA) of crystalline Compound A tosylate salt is shown in FIG. 8. The graph of the thermogravimetric analysis (TGA) of crystalline Form 2 of Compound A is shown in FIG. 13. The graph of the thermogravimetric analysis (TGA) of crystalline Form 4 of Compound A is shown in FIG. 17. The graph of the thermogravimetric analysis (TGA) of crystalline Form 5 of Compound A is shown in FIG. 20. The graph of the thermogravimetric analysis (TGA) of crystalline Form 6 of Compound A is shown in FIG. 23.

### Example 6

This example demonstrates 1H NMR spectroscopic characterization of the crystalline forms of Compound A in accordance with an embodiment of the invention.

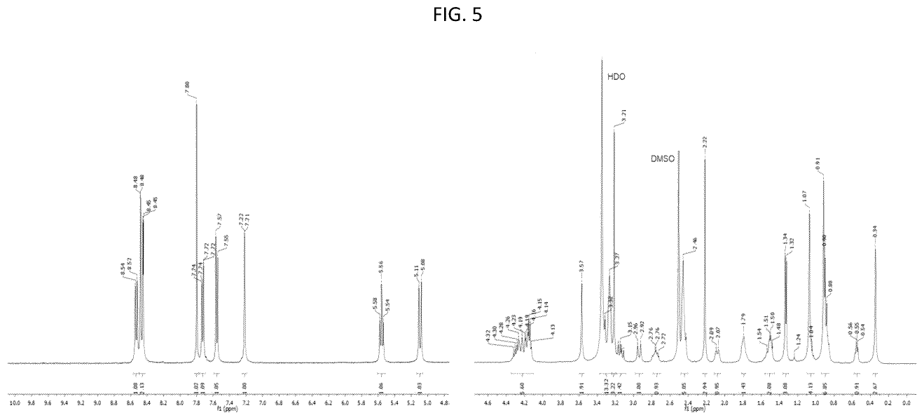

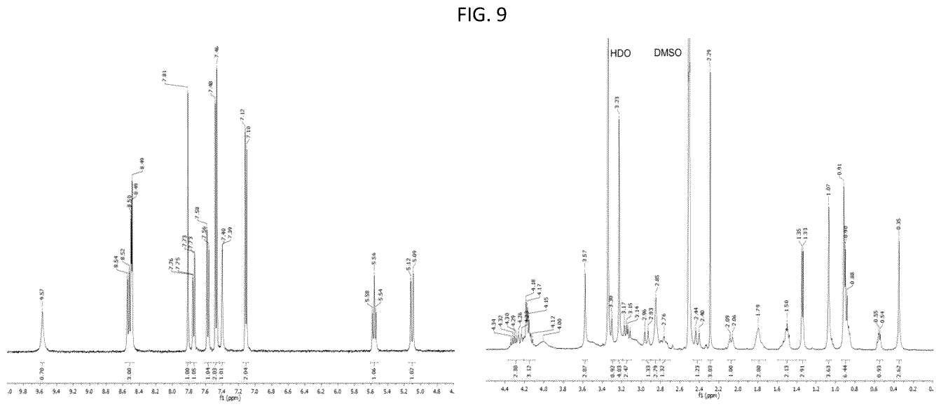

The ¹H NMR spectra were acquired on a Bruker Avance 400 MHz spectrometer using TopSpin v3.2 software. Each sample was dissolved in DMSO-d6 and the resulting solution was transferred into a 5-mm NMR tube for subsequent data acquisition. Data collection parameters are shown in Table 7. The spectrum was processed using the program MNova and referenced to the chemical shift of the residual protons in DMSO-d6. The 1H NMR spectrum of crystalline Compound A free base is shown in FIG. 5. The ¹H NMR spectrum of crystalline Compound A tosylate salt is shown in FIG. 9.

TABLE 7
NMR Data Collection Parameters.

| | | |
|---|---|---|
| Spectral frequency | 400.18 | MHz |
| Spectral width | 6009.6 | Hz |
| Pulse width | 10.22 | μsec |
| Relaxation delay | 4 | μsec |
| Acquisition time | 4 | μsec |

| | |
|---|---|
| Number of scans | 32 |

### Example 7

This example demonstrates a preparation of the crystalline form of Compound A tosylate salt in accordance with an embodiment of the invention.

A heterogeneous mixture of 19.7 mg (24.3 mmol) of Compound A, 4.0 mg (21.0 mmol) of 4-toluenesulfonic acid monohydrate, and 1 mL of acetonitrile was stirred at ambient temperature overnight, then placed on a hot plate set at 40° C. and stirred for about two days. The mixture was allowed to cool to ambient temperature and centrifuged. The liquid layer was decanted and the remaining solid was allowed to dry in the air to give crystalline form of Compound A tosylate salt.

### Example 8

This example demonstrates X-Ray Powder Diffraction (XRPD) characterization of the crystalline Form 1 of Compound A tosylate salt in accordance with an embodiment of the invention.

XRPD measurements were carried out using one of three Rigaku Smart-Lab X-ray diffraction systems. Each was configured for Bragg-Brentano reflection geometry using a line source X-ray beam. The Bragg-Brentano geometry was controlled by passive divergence and receiving slits with the sample itself acting as the focusing component for the optics. Powder samples were prepared in low-background Si holders using light manual pressure to keep the sample surfaces flat and level with the reference surface of the sample holder. Diffractometer specifications and data collection parameters for diffraction system 1 (file prefix RX1) are shown in Table 8. Diffractometer specifications and data collection parameters for diffraction system 2 (file prefix RX3) are shown in Table 9. Diffractometer specifications and data collection parameters for diffraction system 3 (file prefix RX4) are shown in Table 10. The X-ray powder diffractogram of crystalline Compound A tosylate salt is shown in FIG. 6. In an essentially pure material of crystalline Compound A tosylate salt, peaks can be observed at angles of refraction 20 as set forth in Table 9.

TABLE 8
Diffractometer System 1 Specifications
and Data Collection Parameters.

| Parameter | Value |
|---|---|
| Geometry | Reflection |
| Tube Anode | Cu Kα1 |
| Tube Type | Long Fine Focus |
| Tube Voltage (kV) | 40 |
| Tube Current (mA) | 44 |
| Detector | D/teX Ultra PSD^{a} |
| Monochromatization | Kβ Filter |
| Incident Slit (°) | ⅓ |
| Receiving Slit 1 (mm) | 18 |
| Receiving Slit 2 (mm) | open |
| Start Angle (°2θ) | 2 |
| End Angle (°2θ) | 40 |
| Step Size (°2θ) | 0.02 |
| Scan Speed (°2θ/min) | 6 |
| Spinning (rpm) | 11 |
| Sample Holder | low-background Si |

^{a}PSD = position sensitive detector

TABLE 9
Diffractometer System 2 Specifications
and Data Collection Parameters.

| Parameter | Value |
|---|---|
| Geometry | Reflection |
| Tube Anode | Cu Kα1 |
| Tube Type | Long Fine Focus |
| Tube Voltage (kV) | 40 |
| Tube Current (mA) | 44 |
| Detector | D/teX Ultra PSD^{a} |
| Monochromatization | Kβ Filter |
| Incident Slit (°) | ⅓ |
| Receiving Slit 1 (mm) | 8 |
| Receiving Slit 2 (mm) | 13 |
| Start Angle (°2θ) | 2 |
| End Angle (°2θ) | 40 |
| Step Size (°2θ) | 0.02 |
| Scan Speed (°2θ/min) | 6 |
| Spinning (rpm) | 17 |
| Sample Holder | low-background Si |

^{a}PSD = position sensitive detector

TABLE 10
Diffractometer System 3 Specifications
and Data Collection Parameters.

| Parameter | Value |
|---|---|
| Geometry | Reflection |
| Tube Anode | Cu Kα1 |
| Tube Type | Long Fine Focus |
| Tube Voltage (kV) | 40 |
| Tube Current (mA) | 44 |
| Detector | HyPix3000 |
| Monochromatization | Focusing mirror |
| Incident Slit (°) | ⅓ |
| Receiving Slit 1 (mm) | 8 |
| Receiving Slit 2 (mm) | 20 |
| Start Angle (°2θ) | 2 |
| End Angle (°2θ) | 40 |
| Step Size (°2θ) | 0.02 |
| Scan Speed (°2θ/min) | 6 |
| Spinning (rpm) | 11 |
| Sample Holder | low-background Si |

TABLE 11
X-ray powder diffraction peaks of crystalline
Compound A tosylate salt.

| Position [2θ°] | Relative Intensity |
|---|---|
| 5.9 | 39 |
| 9.4 | 11 |
| 9.8 | 20 |
| 10.2 | 20 |
| 10.6 | 17 |
| 11.0 | 23 |
| 11.5 | 9 |
| 11.7 | 9 |
| 12.6 | 24 |
| 12.8 | 41 |
| 13.1 | 10 |
| 13.5 | 17 |
| 14.2 | 20 |
| 14.5 | 6 |
| 15.0 | 12 |
| 15.5 | 10 |
| 16.1 | 100 |
| 16.4 | 32 |
| 17.2 | 67 |
| 17.4 | 13 |
| 17.6 | 13 |
| 18.1 | 38 |
| 18.4 | 58 |
| 19.4 | 32 |
| 19.6 | 21 |
| 19.9 | 28 |
| 20.4 | 24 |
| 20.8 | 36 |
| 21.1 | 18 |
| 21.4 | 24 |
| 21.8 | 12 |
| 22.3 | 29 |
| 22.5 | 40 |
| 23.0 | 17 |
| 23.4 | 23 |
| 23.9 | 40 |
| 24.2 | 16 |
| 24.6 | 20 |
| 25.2 | 16 |
| 25.6 | 7 |
| 26.1 | 17 |
| 27.1 | 34 |
| 27.9 | 9 |
| 28.4 | 14 |
| 28.9 | 11 |
| 29.5 | 10 |
| 30.0 | 8 |
| 30.1 | 7 |
| 31.2 | 7 |
| 31.6 | 8 |
| 32.0 | 7 |
| 32.7 | 8 |
| 33.1 | 7 |
| 33.5 | 8 |
| 33.8 | 10 |
| 34.2 | 6 |
| 34.8 | 8 |
| 35.2 | 6 |
| 35.6 | 9 |
| 37.1 | 7 |
| 38.0 | 6 |
| 38.8 | 6 |
| 39.1 | 7 |

### Example 9

This example demonstrates dynamic vapor sorption (DVS) characterization of the crystalline form of Compound A tosylate salt in accordance with an embodiment of the invention.

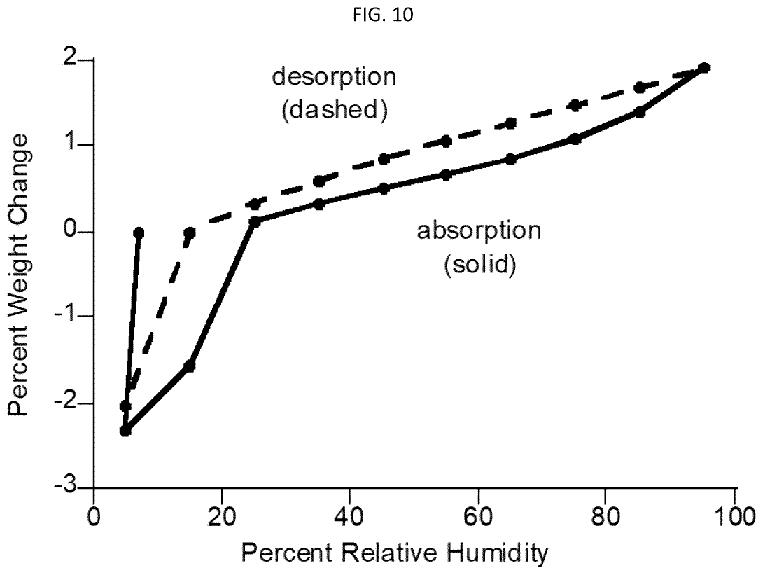

The DVS analysis was carried out on a TA Instruments Q5000 sorption analyzer. The instrument was calibrated with standard weights and a sodium bromide standard for humidity. A portion of the sample was weighed into a metal-coated, quartz pan. Analysis was at 25° C. with a maximum equilibration time of 60 minutes in 10% relative humidity (RH) steps from 5 to 95% RH (adsorption cycle) and from 95 to 5% RH (desorption cycle). Data collection was performed using Advantage for Q Series version 5.5.23. The DVS graph of Compound A tosylate salt is shown in FIG. 10.

### Example 10

This example demonstrates exemplary methods of preparing and characterizing crystalline Form 2 of Compound A.

Exemplary methods of preparing crystalline Form 2 of Compound A include: (1) equilibration of amorphous Compound A in isopropyl acetate or isopropyl acetate; (2) reverse addition of anti-solvent using acetone and methyl tert-butyl ether (MTBE); and (3) equilibration of amorphous Compound A under a temperature cycle.

In the first method, about 30 mg of amorphous Compound A was dissolved in either isopropyl acetate or isopropyl alcohol and stirred at 25° C. for 1 week. The suspension was filtered through a 0.45 μm nylon membrane filter by centrifugation at 14,000 rpm, resulting in crystalline Form 2.

In the second method, about 50 mg of amorphous Compound A was dissolved in a minimal amount of acetone to form a clear solution at ambient temperature (e.g., about 20-25° C.). To this solution, MTBE was added until a large amount of solids precipitated (about 1:4 acetone/MTBE v/v). The solids were collected by centrifugation through a 0.45 μm nylon membrane filter at 14,000 rpm to obtain crystalline Form 2.

In the third method, about 30 mg of amorphous Compound A was equilibrated in 0.1-0.3 mL of isopropyl acetate under a temperature cycle between 5° C. to 50° C. at a heating/cooling rate of 0.1° C./min for 10 cycles. The equilibration was executed with a stirring bar on a magnetic stirring plate at a rate of 300-400 rpm. The resulting suspension was filtered through a 0.45 μm nylon membrane filter by centrifugation at 14,000 rpm to obtain crystalline Form 2.

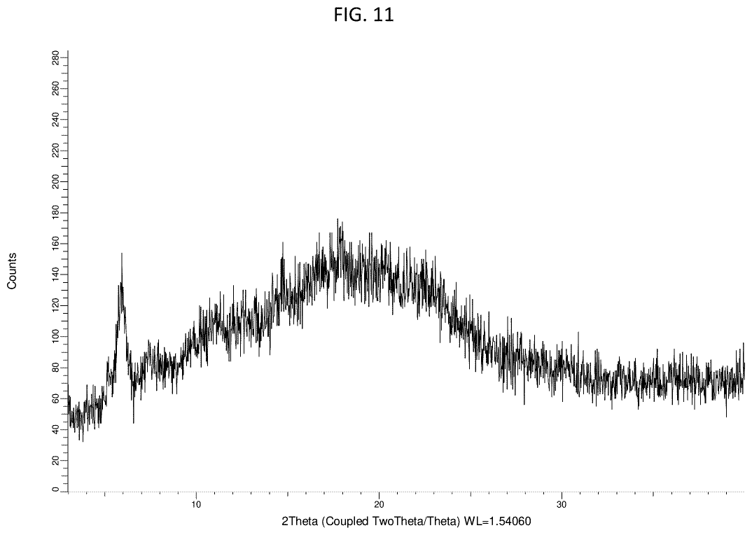

Crystalline Form 2 was characterized by XRPD, DSC, and TGA. By XRPD, Form 2 had low crystallinity (FIG. 11). In an essentially pure material of crystalline Form 2, peaks can be observed at angles of refraction 20 as set forth in Table 12. By DSC, Form 2 exhibited three desolvation/dehydration peaks at Tₒₙₛₑₜ of 43.5° C. with an enthalpy of about 58 J/g, Tₒₙₛₑₜ of 131.5° C. with an enthalpy of about 6 J/g, and Tₒₙₛₑₜ of 180.7° C. with an enthalpy of about 3 J/g, then followed by a melting peak at Tₒₙₛₑₜ of 205.7° C. combined with decomposition (FIG. 12). By TGA, Form 2 exhibited about 5.1% weight loss at about 80° C. and 2.3% weight loss from 80° C. to 160° C. (FIG. 13).

TABLE 12
X-ray powder diffraction peaks of
crystalline Form 2 of Compound A.

| Position [2θ°] | Relative Intensity |
|---|---|
| 5.9 | 100.00 |
| 14.7 | 38.30 |

### Example 11

This example demonstrates exemplary methods of preparing and characterizing crystalline Form 3 of Compound A.

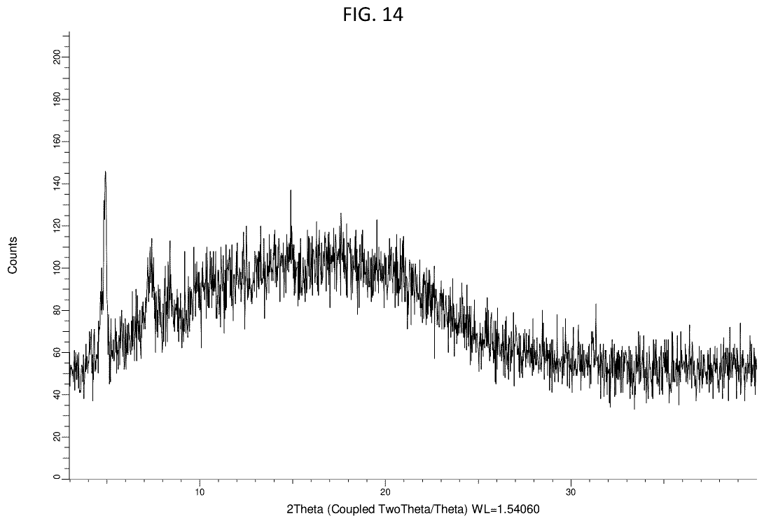

About 30 mg of amorphous Compound A was dissolved in 0.1-0.3 mL of isopropyl alcohol and stirred at 25° C. for 1 week. The suspension was filtered through a 0.45 μm nylon membrane filter by centrifugation at 14,000 rpm, resulting in crystalline Form 3. Crystalline Form 3 was characterized by XRPD. By XRPD, Form 3 had low crystallinity (FIG. 14). In an essentially pure material of crystalline Form 3, peaks can be observed at angles of refraction 20 as set forth in Table 13.

TABLE 13
X-ray powder diffraction peaks of
crystalline Form 3 of Compound A.

| Position [2θ°] | Relative Intensity |
|---|---|
| 4.9 | 100.00 |
| 7.4 | 61.40 |

### Example 12

This example demonstrates exemplary methods of preparing and characterizing crystalline Form 4 of Compound A.

Exemplary methods of preparing crystalline Form 4 of Compound A include: (1) equilibration of amorphous Compound A in toluene or ethyl acetate/heptane; (2) equilibration of amorphous Compound A under a temperature cycle; and (3) equilibration of Form 1 in ethyl acetate/heptane.

In the first method, about 30 mg of amorphous Compound A was dissolved in 0.1-0.3 mL of toluene and stirred at 25° C. for 1 week. The suspension was filtered through a 0.45 μm nylon membrane filter by centrifugation at 14,000 rpm, resulting in crystalline Form 4.

In the second method, about 30 mg of amorphous Compound A was equilibrated in 0.1-0.3 mL of toluene under a temperature cycle between 5° C. to 50° C. at a heating/cooling rate of 0.1° C./min for 10 cycles. The equilibration was executed with a stirring bar on a magnetic stirring plate at a rate of 300-400 rpm. The resulting suspension was filtered through a 0.45 μm nylon membrane filter by centrifugation at 14,000 rpm to obtain crystalline Form 4.

In the third method, about 30 mg of crystalline Form 1 of Compound A was dissolved in 0.1-0.3 mL of 1:1 (v:v) ethyl acetate/heptane and stirred at 25° C. for 1 week. The suspension was filtered through a 0.45 μm nylon membrane filter by centrifugation at 14,000 rpm, resulting in crystalline Form 4.

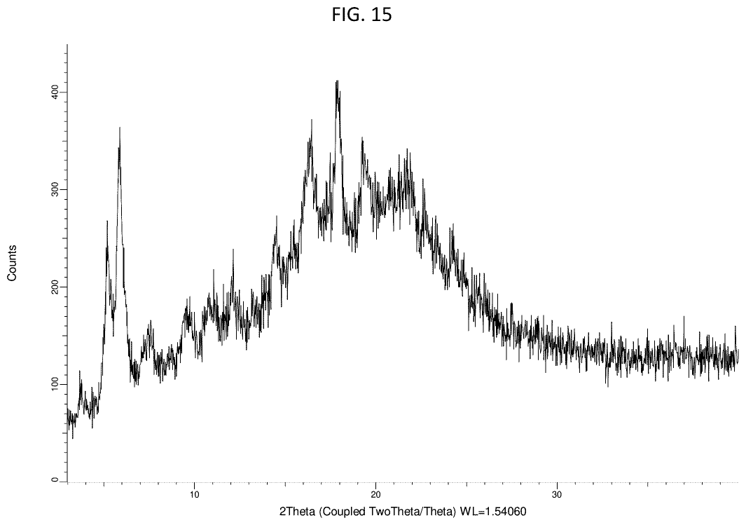

Crystalline Form 4 was characterized by XRPD, DSC, and TGA. By XRPD, Form 4 had low crystallinity (FIG. 15). In an essentially pure material of crystalline Form 4, peaks can be observed at angles of refraction 20 as set forth in Table 14. By DSC, Form 4 exhibited a dehydration peak at Tₒₙₛₑₜ of 30.9° C. with an enthalpy of 63 J/g (FIG. 16). There is no obvious melting after dehydration. By TGA, Form 4 exhibited 4.6% weight loss at 80° C. and 2.5% weight loss from 80° C. to 160° C. (FIG. 17).

TABLE 14
X-ray powder diffraction peaks of
crystalline Form 4 of Compound A.

| Position [2θ°] | Relative Intensity |
|---|---|
| 5.2 | 51.10 |
| 5.9 | 100.00 |
| 12.1 | 36.60 |
| 14.5 | 32.20 |
| 16.4 | 54.90 |
| 17.9 | 61.80 |
| 19.4 | 40.40 |

### Example 13

This example demonstrates exemplary methods of preparing and characterizing crystalline Form 5 of Compound A, which was identified as a hydrate.

Exemplary methods of preparing crystalline Form 5 of Compound A include: (1) equilibration experiments using Form 1; and (2) precipitation by the addition of anti-solvent experiments using Form 1.

In the first method, about 25 mg of crystalline Form 1 of Compound A was equilibrated in 0.1-0.3 mL of either 1:2 (v:v) isopropyl alcohol/water or 1:2 (v:v) acetone/water at 50° C. for 4 days with a stirring bar on a magnetic stirring plate at a rate of 300-400 rpm. The resulting suspension was filtered through a 0.45 μm nylon membrane filter by centrifugation at 14,000 rpm to obtain crystalline Form 5.

In the second method, about 50 mg of crystalline Form 1 of Compound A was dissolved in a minimal amount of isopropyl alcohol at ambient temperature (about 20-25° C.). To this solution, water was added slowly until a large amount of solids precipitated out (1:2 v:v isopropyl alcohol/water). The precipitate was collected by centrifugation through a 0.45 μm nylon membrane filter at 14,000 rpm to obtain crystalline Form 5.

Crystalline Form 5 was characterized by XRPD, DSC, and TGA. By XRPD, Form 5 had high crystallinity (FIG. 18). In an essentially pure material of crystalline Form 5, peaks can be observed at angles of refraction 20 as set forth in Table 15. By DSC, Form 5 exhibited a dehydration peak at Tₒₙₛₑₜ of 32.1° C. with an enthalpy of 48 J/g, a recrystallization peak at Tₒₙₛₑₜ of 81.2° C., and a melting peak at Tₒₙₛₑₜ of 172.5° C. combined with recrystallization (FIG. 19). TGA shows 4.8% weight loss at 150° C. (FIG. 20).

TABLE 15
X-ray powder diffraction peaks of
crystalline Form 5 of Compound A.

| Position [2θ°] | Relative Intensity |
|---|---|
| 3.5 | 2.10 |
| 5.2 | 46.60 |
| 6.8 | 3.30 |
| 8.4 | 9.60 |
| 8.6 | 26.60 |
| 9.1 | 12.30 |
| 9.9 | 17.50 |
| 10.3 | 24.40 |
| 10.6 | 27.40 |
| 11.6 | 18.60 |
| 11.9 | 3.50 |
| 12.2 | 29.00 |
| 12.4 | 16.10 |
| 13.2 | 10.00 |
| 13.5 | 5.70 |
| 14.3 | 100.00 |
| 14.6 | 18.50 |
| 14.7 | 30.00 |
| 15.0 | 10.10 |
| 15.4 | 23.40 |
| 16.3 | 33.50 |
| 16.8 | 68.40 |
| 17.0 | 49.20 |
| 17.1 | 30.40 |
| 17.5 | 19.20 |
| 18.2 | 31.80 |
| 18.4 | 13.90 |
| 18.8 | 15.30 |
| 19.0 | 27.80 |
| 19.9 | 2.10 |
| 20.3 | 35.30 |
| 20.5 | 49.70 |
| 20.8 | 24.30 |
| 21.0 | 21.50 |
| 21.5 | 11.20 |
| 21.8 | 7.70 |
| 22.3 | 5.60 |
| 22.6 | 7.80 |
| 23.0 | 3.90 |
| 23.3 | 22.60 |
| 23.6 | 7.40 |
| 23.7 | 16.00 |
| 24.0 | 30.00 |
| 24.4 | 21.20 |
| 24.8 | 9.00 |
| 25.0 | 18.60 |
| 25.8 | 19.00 |
| 26.3 | 4.00 |
| 26.8 | 3.80 |
| 27.1 | 13.80 |
| 27.5 | 4.00 |
| 27.7 | 4.40 |
| 28.9 | 4.80 |
| 29.4 | 3.70 |
| 29.8 | 6.00 |
| 30.2 | 4.20 |
| 30.7 | 7.80 |
| 31.1 | 4.10 |
| 31.3 | 4.10 |
| 31.8 | 3.00 |
| 32.0 | 2.90 |
| 32.4 | 2.40 |
| 32.9 | 2.50 |
| 33.7 | 4.30 |
| 35.6 | 3.40 |
| 36.1 | 2.50 |
| 36.9 | 2.80 |
| 39.3 | 3.00 |

### Example 14

This example demonstrates exemplary methods of preparing and characterizing crystalline Form 6 of Compound A.

About 100 mg of crystalline Form 1 of Compound A was equilibrated in 0.5 mL of 1:1 (v:v) ethyl acetate/heptane at 25° C. About 2 mg of Form 4 seeds were added to the mixture. After stirring the mixture at 25° C. for two days, the resulting solids were collected by vacuum filtration to obtain crystalline Form 5.

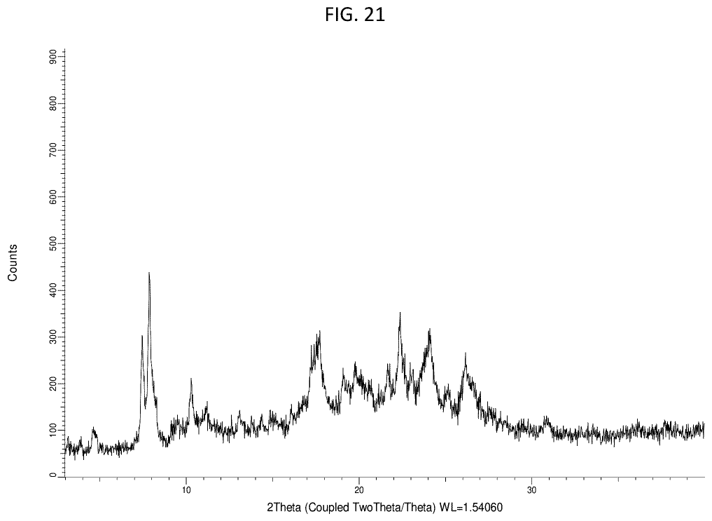

Crystalline Form 6 was characterized by XRPD, DSC, and TGA. By XRPD, Form 6 had low crystallinity (FIG. 21). In an essentially pure material of crystalline Form 6, peaks can be observed at angles of refraction 20 as set forth in Table 16. By DSC, Form 6 exhibited a desolvation peak at Tₒₙₛₑₜ of 53.3° C. with an enthalpy of 44 J/g and an endothermic peak at Tₒₙₛₑₜ of 166.4° C. with an enthalpy of 4 J/g (FIG. 22). By TGA, Form 6 exhibited 6.7% weight loss at 150° C. (FIG. 23).

TABLE 16
X-ray powder diffraction peaks of
crystalline Form 6 of Compound A.

| Position [2θ°] | Relative Intensity |
|---|---|
| 4.7 | 8.30 |
| 7.5 | 68.40 |
| 7.9 | 100.00 |
| 10.3 | 38.90 |
| 11.2 | 11.50 |
| 13.1 | 13.70 |
| 17.4 | 29.90 |
| 17.7 | 49.00 |
| 19.1 | 16.60 |
| 19.8 | 27.80 |
| 20.6 | 10.70 |
| 21.7 | 24.30 |
| 22.4 | 55.90 |
| 23.1 | 24.80 |
| 24.1 | 44.10 |
| 25.2 | 13.30 |
| 26.2 | 41.50 |

### Example 15

This example demonstrates exemplary methods of preparing and characterizing crystalline Form 7 of Compound A.

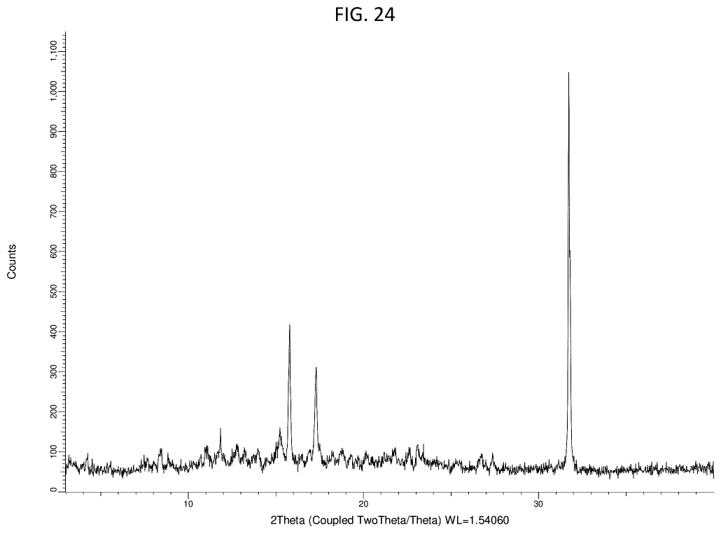

About 8 mg of crystalline Form 1 of Compound A was weighed into an 8-mL glass vial. To this vial, 4 mL of either FeSSIF-V1 (pH 5.0) or FaSSIF-V1 (pH 6.5) was added. The solution was stirred at 37° C. at 400 rpm for 0.5 h, 2 h, and 24 h and then centrifuged at 37° C. at 14,000 rpm for 5 min to obtain crystalline Form 7. By XRPD, Form 7 had low crystallinity (FIG. 24). In an essentially pure material of crystalline Form 7, peaks can be observed at angles of refraction 20 as set forth in Table 17.

TABLE 17
X-ray powder diffraction peaks of
crystalline Form 7 of Compound A.

| Position [2θ°] | Relative Intensity |
|---|---|
| 5.4 | 1.50 |
| 8.4 | 4.20 |
| 8.4 | 3.00 |
| 10.7 | 1.80 |
| 11.1 | 4.60 |
| 11.5 | 2.60 |
| 11.8 | 6.40 |
| 12.8 | 4.10 |
| 13.2 | 2.10 |
| 14.0 | 3.40 |
| 15.2 | 6.60 |
| 15.8 | 27.60 |
| 16.9 | 2.20 |
| 17.3 | 18.70 |
| 19.3 | 1.80 |
| 20.2 | 2.50 |
| 21.2 | 3.10 |
| 21.8 | 3.20 |
| 22.7 | 2.70 |
| 23.1 | 4.10 |
| 23.4 | 2.70 |
| 24.9 | 2.50 |
| 26.7 | 3.30 |
| 27.4 | 3.70 |
| 31.7 | 100.00 |
| 39.2 | 2.90 |

### Example 16

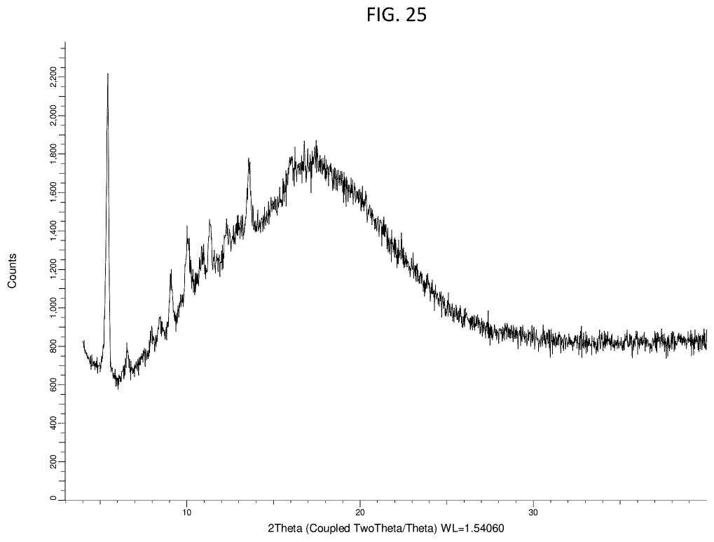

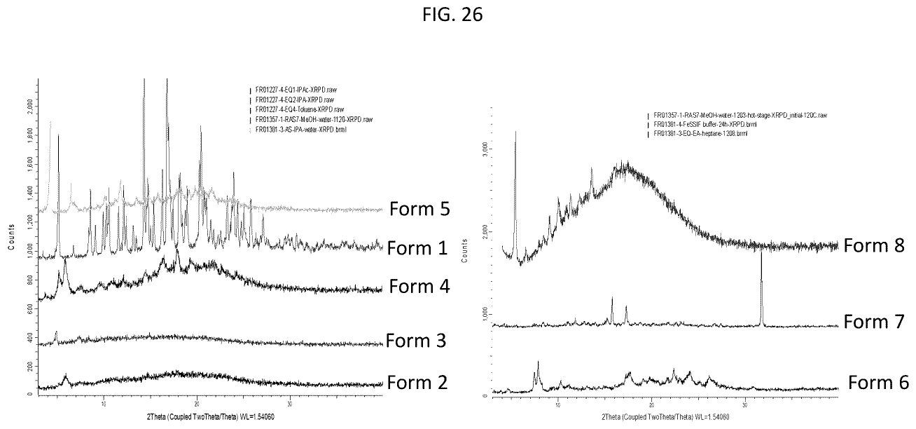

This example demonstrates exemplary methods of preparing and characterizing crystalline Form 8 of Compound A. Crystalline Form 8 was obtained by heating crystalline Form 1 of Compound A to 120° C. at 0% RH. When the RH was increased to 40%, crystalline Form 8 converted back to crystalline Form 1. By XRPD, Form 8 had low crystallinity (FIG. 25). In an essentially pure material of crystalline Form 8, peaks can be observed at angles of refraction 20 as set forth in Table 18. FIG. 26 shows an overlay of XRPDs of crystalline Forms 1-8 of Compound A.

TABLE 18
X-ray powder diffraction peaks of
crystalline Form 8 of Compound A.

| Position [2θ°] | Relative Intensity |
|---|---|
| 5.4 | 100.00 |
| 6.6 | 7.00 |
| 8.0 | 8.60 |
| 8.5 | 8.00 |
| 9.1 | 23.90 |
| 10.1 | 25.30 |
| 10.9 | 18.60 |
| 11.3 | 26.70 |
| 12.3 | 19.30 |
| 13.6 | 44.00 |
| 16.0 | 37.80 |
| 17.5 | 35.30 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent, or patent application was specifically and individually indicated to be incorporated by reference in its entirety.

1. A crystalline solid form of Compound A: 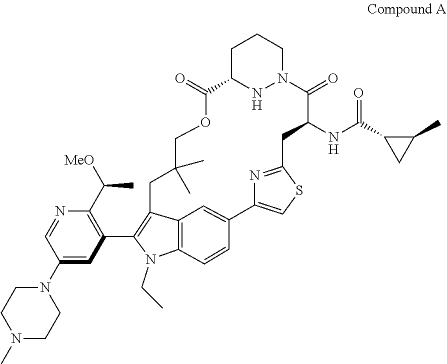
or a solvate thereof.

2. The crystalline solid form of claim 1, wherein Compound A or the solvate thereof is selected from Form 1, Form 2, Form 3, Form 4, Form 5, Form 6, Form 7, or Form 8.

3. The crystalline solid form of claim 1 or 2, wherein Compound A or the solvate thereof is Form 1.

4. The crystalline solid form of claim 1 or 2, wherein Compound A or the solvate thereof has Form 5.

5. A crystalline hydrate of Compound A.

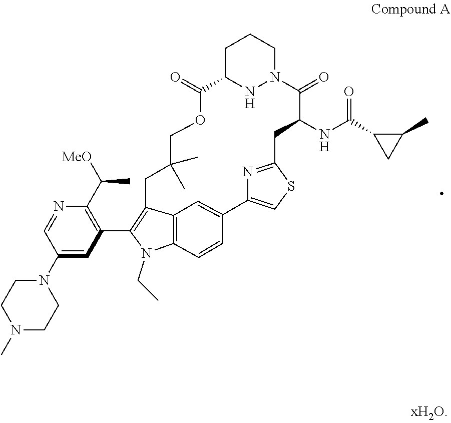

6. The crystalline solid form of claim 3, or a solvate thereof, having at least one peak at diffraction angle 2θ (°) of 5.2±0.5, 8.4±0.5, or 10.0±0.5 as measured by X-ray diffractometry by irradiation with Cu Kα X-rays or calculated from X-ray diffractometry.

7. A crystalline Form 5 of Compound A: 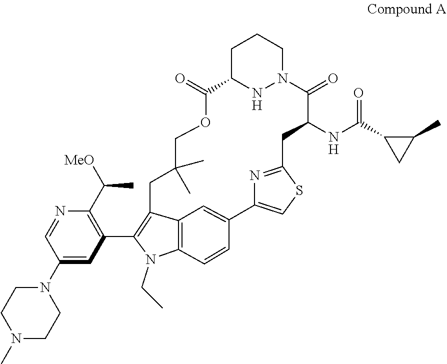
or a solvate thereof, having at least one peak at diffraction angle 2θ (°) of 14.3±0.5, 16.8±0.5, or 20.5±0.5 as measured by X-ray diffractometry by irradiation with Cu Kα X-rays or calculated from X-ray diffractometry.

8. A crystalline solid form of a tosylate salt of Compound A or a solvate thereof.

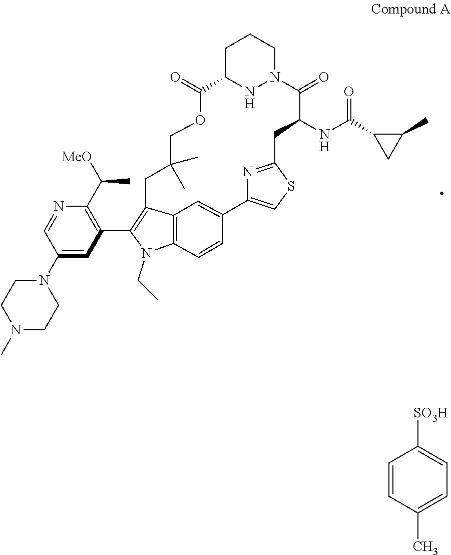

9. The crystalline solid form of claim 8, or a solvate thereof, having at least one peak at diffraction angle 2θ (°) of 5.9±0.5, 9.4±0.5, or 9.8±0.5 as measured by X-ray diffractometry by irradiation with Cu Kα X-rays or calculated from X-ray diffractometry.

10. A pharmaceutical composition comprising the crystalline forms of Compound A of claim 2, or a solvate thereof, and a pharmaceutically acceptable carrier or excipient.

11. A method of making a crystalline Form 1 of Compound A, 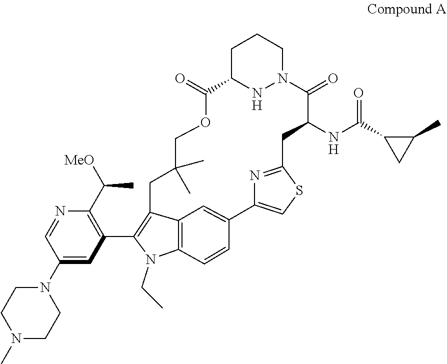
or a solvate thereof, comprising preparing a slurry of Compound A in a suitable solvent or buffer to precipitate crystalline solids of Form 1, isolating the crystalline solids of Form 1 from the slurry, and drying the crystalline solids of Form 1.

12. A method of making a crystalline Form 5 of Compound A, 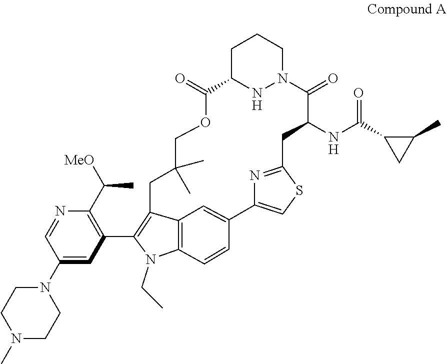
a solvate thereof, comprising preparing a slurry of crystalline Form 1 of Compound A in a suitable solvent to precipitate crystalline solids of Form 5, isolating the crystalline solids of Form 5 from the slurry, and drying the crystalline solids of Form 5.

13. A method of making a crystalline Form 5 of Compound A, 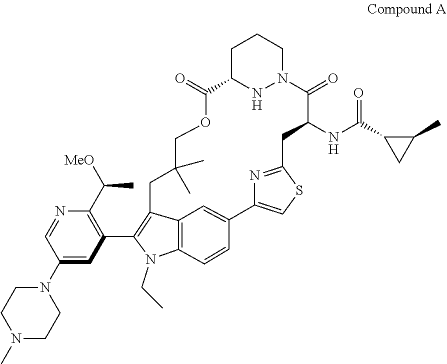
or a solvate thereof, comprising preparing a mixture of crystalline Form 1 of Compound A in isopropyl alcohol, precipitating crystalline solids of Form 5 by the addition of water, isolating the crystalline solids of Form 5 from the slurry, and drying the crystalline solids of Form 5.

14. A method of making a crystalline form of a tosylate salt Compound A, 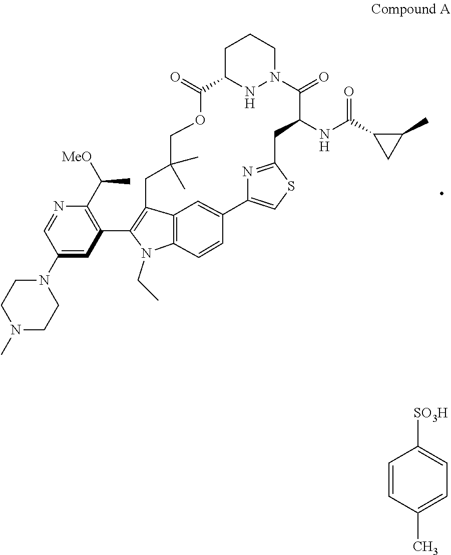
or a solvate thereof, comprising preparing a mixture of Compound A and 4-toluenesulfonic acid in a suitable solvent or buffer to precipitate crystalline solids of the tosylate salt of Compound A, isolating the crystalline solids of the tosylate salt of Compound A from the mixture, and drying the crystalline solids of the tosylate salt of Compound A.

15. A method of treating cancer in a subject in need thereof, the method comprising administering to the subject a therapeutically effective amount of a crystalline form of Compound A of claim 2, or a solvate thereof.

16. A method of treating a Ras protein-related disorder in a subject in need thereof, the method comprising administering to the subject a therapeutically effective amount of a crystalline form of Compound A of claim 2, or a solvate thereof.

17. A method of inhibiting a Ras protein in a cell, the method comprising contacting the cell with an effective amount of a crystalline form of Compound A of claim 2, or a solvate thereof.
